(19) United States
(12) Patent Application Publication
(10) Pub. No.: US 2026/0207278 A1
(43) Pub. Date: Jul. 23, 2026

(54) ACTUATOR FOR ROBOTIC SYSTEM

(71) Applicant: Auris Health, Inc., Santa Clara, CA (US)

(72) Inventor: Bryan James Culver, Mountain View, CA (US)

(21) Appl. No.: 19/567,911

(22) Filed: Mar. 16, 2026

Related U.S. Application Data
(63) Continuation of application No. PCT/IB2025/059450, filed on Sep. 16, 2025.
(60) Provisional application No. 63/707,191, filed on Oct. 14, 2024.
(60) Provisional application No. 63/878,422, filed on Sep. 9, 2025.

(51) Int. Cl. A61B 34/30; A61B 90/00; B25J 9/10

(52) U.S. Cl. CPC A61B 34/30; A61B 90/06; B25J 9/102; A61B 2090/066

(57) ABSTRACT

Among other things, an actuator for a surgical tool driver is disclosed. The actuator includes an internal ring gear including a first plurality of involute teeth and centered relative to a central axis. The actuator also includes a cycloid disc positioned eccentrically relative to the central axis and including a second plurality of involute teeth and a first plurality of bushing surfaces. The actuator further includes a rotor configured to drive eccentric movement of the cycloid disc relative to the central axis. The actuator also includes an eccentric coupler including an eccentric coupler input having a second plurality of bushing surfaces configured to selectively engage the first plurality of bushing surfaces to thereby convert eccentric movement of the cycloid disc relative to the central axis into rotation of the eccentric coupler about the central axis. The eccentric coupler includes an eccentric coupler output configured to operatively engage a drive output.

### PRIORITY

This application claims the benefit of U.S. Pat. App. No. 63/878,422, entitled “Actuator for Robotic System,” filed Sep. 9, 2025, and U.S. Pat. App. No. 63/707,191, entitled “Actuator for Robotic System,” filed Oct. 14, 2024, the disclosures of which are incorporated by reference herein.

### BACKGROUND

Minimally invasive medical procedures, such as laparoscopy, endoscopy, and robotically-assisted surgery, are increasingly used for the diagnosis or treatment of a variety of patient conditions. These techniques are attractive for their potential to minimize trauma to the patient, reduce recovery times, enhance surgeon precision, or facilitate new surgical approaches that may not be possible with traditional technologies. Such procedures can involve elongate instruments introduced through small incisions or natural orifices on a patient's body to reach an anatomical site. These instruments are then manipulated to observe or interact with target anatomy within the patient using the tips of these instruments. A surgeon may control these instruments while observing a real-time camera feed of the anatomical site.

### BRIEF DESCRIPTION OF DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an example of a surgical system.

FIG. 2 depicts an example of a physician console from the surgical system of FIG. 1.

FIG. 3 depicts an example of the surgical system of FIG. 1 configured for combined endoscopic and laparoscopic surgery.

FIG. 4 depicts an example of an anatomical site from the surgical system of FIG. 3.

FIG. 5 depicts an example of a physician console for use with the surgical system of FIG. 3, configured for displaying endoscopic and laparoscopic views.

FIG. 6 depicts an example of a surgical robot for use with the surgical system of FIG. 3, configured for manipulating rigid and flexible instruments for combined endoscopic and laparoscopic surgery.

FIG. 7 depicts an example of a robotic manipulator for use with the surgical robot of FIG. 6, configured for manipulating a rigid instrument.

FIG. 8 depicts an example of a robotic manipulator for use with the surgical robot of FIG. 6, configured for manipulating a flexible instrument.

FIG. 9A depicts an example of a distal assembly for use with the robotic manipulators of FIGS. 7-8, with a tool driver and an instrument in a coupled configuration.

FIG. 9B depicts the distal assembly of FIG. 9A, with the tool driver and the instrument in a decoupled configuration.

FIG. 10 depicts a perspective view of another example of a tool driver that may be used in place of the tool driver of FIGS. 9A-9B.

FIG. 11 depicts a perspective view of an actuator of the tool driver of FIG. 10.

FIG. 12 depicts an exploded perspective view of the actuator of FIG. 11, showing a housing assembly, a rotor assembly, a cycloid disc, an eccentric coupler assembly, and an output puck of the actuator.

FIG. 13 depicts a side cross-sectional view of the actuator of FIG. 11, taken along line 13-13 in FIG. 11.

FIG. 13A depicts a partial side cross-sectional view of the actuator of FIG. 11, showing a rotor shaft of the rotor assembly in a respective first rotational position relative to a central axis of the rotor assembly, in which the cycloid disc is offset toward a first side of the central axis such that an eccentric coupler input of the eccentric coupler assembly and the output puck are each in respective first rotational positions.

FIG. 13B depicts a partial side cross-sectional view of the actuator of FIG. 11, showing the rotor shaft in a respective second rotational position relative to the central axis, in which the cycloid disc is offset toward a second side of the central axis such that the eccentric coupler input and the output puck are each in respective second rotational positions.

FIG. 14A depicts a top cross-sectional view of the actuator of FIG. 11, showing the rotor shaft in the respective first rotational position relative to the central axis, in which the cycloid disc is offset toward the first side of the central axis such that the eccentric coupler and the output puck are each in the respective first rotational positions.

FIG. 14B depicts a top cross-sectional view of the actuator of FIG. 11, showing the rotor shaft in the respective second rotational position relative to the central axis, in which the cycloid disc is offset toward the second side of the central axis such that the eccentric coupler and the output puck are each in the respective second rotational positions.

FIG. 15 depicts a perspective view of the housing assembly of FIG. 12.

FIG. 16 depicts an exploded perspective view of the housing assembly of FIG. 12, showing a stator housing bottom, a stator, a stator housing top, a spline plate, a guard shim, a top cover, and a printed circuit assembly of the housing assembly.

FIG. 17 depicts a top plan view of the stator housing bottom of FIG. 16.

FIG. 18 depicts a top plan view of the stator housing top of FIG. 16.

FIG. 19 depicts a top plan view of the spline plate of FIG. 16.

FIG. 20 depicts a top plan view of the top cover of FIG. 16.

FIG. 21 depicts a perspective view of the rotor assembly of FIG. 12.

FIG. 22 depicts an exploded perspective view of the rotor assembly of FIG. 12, showing a rotor, a lower needle bearing, a rotor shaft, a lower ball bearing, a balancing shim, a lower balancing disc, an upper ball bearing, an upper balancing disk, and an upper needle bearing of the rotor assembly.

FIG. 23 depicts a top plan view of the rotor shaft of FIG. 22.

FIG. 24 depicts a side elevational view of the rotor shaft of FIG. 22.

FIG. 25 depicts a perspective view of the cycloid disc of FIG. 12.

FIG. 26 depicts a top plan view of the cycloid disc of FIG. 12.

FIG. 27 depicts a perspective view of the eccentric coupler assembly of FIG. 12.

FIG. 28 depicts an exploded perspective view of the eccentric coupler assembly of FIG. 12, showing a lower ball bearing, an eccentric coupler input, a spacer, an upper ball bearing, an eccentric coupler output, and a compression spring of the eccentric coupler assembly.

FIG. 29 depicts a bottom plan view of the eccentric coupler input of FIG. 28.

FIG. 30 depicts a side elevational view of the eccentric coupler input of FIG. 28.

FIG. 31 depicts a perspective view of an example of a top cover assembly that may be used in place of the top cover of FIG. 16 for providing magnetic torque sensing capabilities.

FIG. 32 depicts an exploded perspective view of the top cover assembly of FIG. 31, showing a top cover and a torque sensing assembly of the top cover assembly.

FIG. 33 depicts a perspective view of the top cover of FIG. 32.

FIG. 34 depicts a top plan view of the top cover of FIG. 32.

FIG. 35 depicts a partial cross-sectional view of an example of a top cover assembly that may be used in place of the top cover of FIG. 16 for providing optical torque sensing capabilities.

FIG. 36 depicts a partial cross-sectional view of another example of a top cover assembly that may be used in place of the top cover of FIG. 16 for providing optical torque sensing capabilities.

FIG. 37 depicts a perspective view of an example of a top cover assembly that may be used in place of the top cover of FIG. 16 for providing capacitive torque sensing capabilities.

FIG. 38 depicts a schematic view of another example of an actuator that may be incorporated into the tool driver of FIGS. 9A-9B, and having torque sensing capabilities.

FIG. 39 depicts a schematic view of another example of an actuator that may be incorporated into the tool driver of FIGS. 9A-9B, and having torque sensing capabilities.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description and appended drawings contain certain examples and configurations of this technology and are not intended to be an exhaustive disclosure of the only configurations in which the technology may be practiced. Other examples, features, aspects, embodiments, and advantages of the technology will be apparent to those skill in the art from this disclosure. As will be realized, the technology described herein is capable of other different and obvious aspects, all without departing from the inventive concepts disclosed herein. Accordingly, the drawings and descriptions should be regarded as illustrative in nature and not restrictive. In some instances, well-known structures and components are not described in detail or are shown in block diagram form to avoid obscuring concepts of this technology.

Minimally invasive procedures such as laparoscopy and endoscopy may allow a physician to control elongate instruments introduced through small incisions or natural orifices on a patient's body to observe or interact with anatomical sites within the patient.

Laparoscopic procedures, for instance, can involve rigid surgical instruments introduced through one or more small incisions on a patient's abdomen. These laparoscopic instruments are manipulated from outside the patient's body through these small incisions to perform various surgical functions. The physician can perform surgical tasks, such as cutting, cauterizing, or grasping while observing a real-time camera feed of the internal anatomical site captured with one of these instruments (a rigid endoscope referred to as “laparoscope”). Because the physician views and interacts with organs or anatomical structures through small ports, rather than directly viewing such anatomical structures through a large incision, laparoscopic procedures are typically less invasive than open surgery.

Endoscopic procedures, for instance, can involve flexible instruments introduced through natural orifices on a patient, such as a mouth or perineal access point. Because these instruments are flexible, they can traverse through a lumen of a patient, which may follow a tortuous path, to reach a target anatomical site. The physician can visualize the anatomy using images obtained from a flexible endoscope to examine or diagnose conditions, or the physician may perform procedural tasks such as taking a sample or applying energy to the target site accessed with the flexible instrument. Because these techniques may involve accessing anatomy through natural orifices, they may be less invasive than laparoscopic techniques while allowing the physician to access or visualize regions of the patient anatomy that may not be readily reachable using laparoscopic techniques, such as the inside of organs like the stomach or colon.

Combining endoscopic and laparoscopic techniques can provide benefits of both approaches, with the flexibility for a physician to view and/or manipulate anatomy from endoscopic or laparoscopic perspectives. Among other things, systems and methods are described herein that facilitate combined use of endoscopic and laparoscopic approaches. In some configurations, a robotic system is provided that allows a single user to interact with a patient anatomy from endoscopic and laparoscopic approaches.

These and other features of the disclosed technology are further described below with respect to examples shown in the figures. It will be appreciated that there are various technical features and concepts disclosed herein which may be practiced independently from each other, in various combinations, or in other contexts beyond the particular examples shown and described with respect to these figures. Accordingly, these examples are explanatory in nature but should not be construed as limiting the scope of the inventive subject matter to the precise examples disclosed.

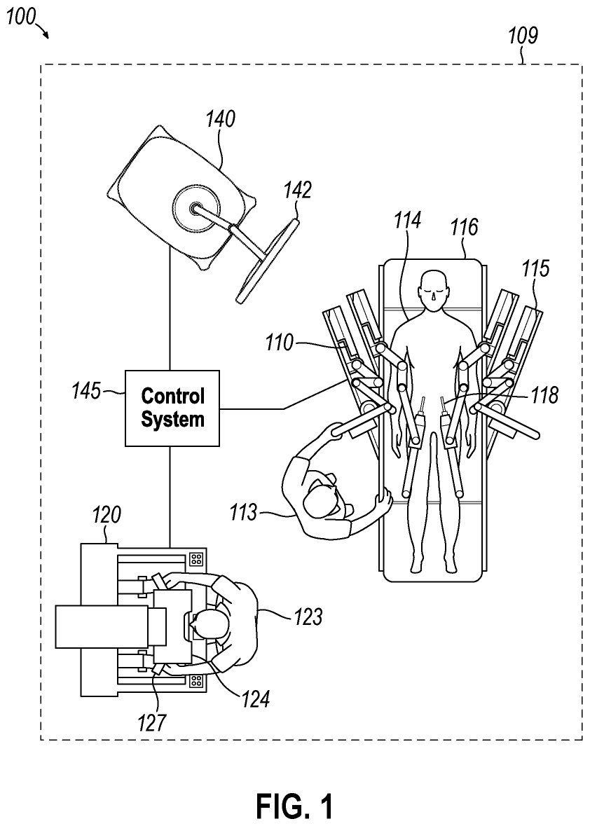

FIG. 1 depicts an example of a surgical system, in accordance with some embodiments. The surgical system 100 can be used to perform a variety of surgical procedures to diagnose and/or treat a patient 114. Examples of procedures include laparoscopy, endoscopy, thoracoscopy, urological procedures, and/or gastrointestinal (GI) procedures. As illustrated, the surgical system 100 is implemented as a robotic surgical system deployed for robotically-assisted procedures.

As seen in FIG. 1, surgical system 100 includes a surgical robot 110, a physician console 120, and a support tower 140. These components are set up in procedure area 109, such as an operating room or an endoscopy suite, and may be used in concert with each other to perform a procedure on patient 114. Components of the surgical system may be coupled physically, communicatively, and/or operatively as appropriate to facilitate operation of the surgical system 100. For instance, any two or more of the surgical robot 110, physician console 120, support tower 140, and/or other components of the surgical system may be interconnected via electrical signal lines, cabling, and/or wireless interconnections. In some configurations, components of the surgical system 100 can be situated in a common room or site. In some configurations, components of the surgical system 100 can be distributed across two or more rooms or sites that are remote from each other, where such components can be communicatively coupled over a network to implement a surgical procedure.

Surgical robot 110 is configured to interact with a patient 114 and perform various tasks. Surgical robot 110 can be actuated based on commands received from physician console 120. Such movements by the robot may be referred to as teleoperation (or telemanipulation), as they involve manipulations that are performed by the robot under human control, rather than fully autonomously. Alternatively, or in combination, surgical robot 110 can be configured to implement one or more tasks fully autonomously or semi-autonomously.

In the illustrated example, surgical robot 110 includes one or more robotic manipulators 115 configured to manipulate one or more instruments 118 (also referred to herein as “tools”). Examples of instruments include graspers, forceps, scissors, scopes, hooks, needle drivers, staplers, biopsy tools, energy delivery instruments, suction devices, irrigation devices, sheaths, biopsy instruments, snares, and various elongate instruments having flexible or rigid shafts that may be inserted into a patient's body. In some instances, an instrument may provide a combination of two or more functions to thereby provide two or more of these instrument types in a single device. Examples of combination instruments include bipolar forceps, handle tissue and deliver energy, suction-irrigators, which provide both suction and irrigation of fluids. Surgical robot 110 can use distal portions of instruments 118 to interact with the patient or perform various procedure tasks, such as manipulating tissue or capturing endoscopic images. In various configurations, the robotic manipulator(s) 115 may be configured to manipulate multiple different types of instruments within a particular procedure and/or across different procedures, allowing the robot to use a variety of instruments to perform a variety of surgical functions. Each of the instruments 118 may be actuatable by the surgical robot 110 in one or more degrees of freedom (DOFs) of the instrument 118, or in some variations, any one of more of the instruments 118 may be non-actuated.

Instruments 118 can be inserted into a body of patient 114 through one or more ports to access an anatomical site within the patient's body. Ports may be formed, for example, by laparoscopic incisions, cannulas, and/or natural orifices on a patient's body to provide an access channel for the instruments 118 to be introduced into the patient's body and advanced to the target anatomical site within the body. In some variations, procedures may involve one or several ports on the patient. In some variations, each port may be used to introduce one or multiple instruments concurrently or sequentially.

The robotic manipulators 115 can each include one or more actuators (e.g., motors) that can be electronically controlled to manipulate the instruments 118. For example, a robotic manipulator 115 can be actuated to control a position of an instrument 118 within the patient's body and/or to actuate mechanisms of the instrument (e.g., to articulate or operate an instrument tip in one or more degrees of freedom). In some variations, surgical robot 110 includes multiple robotic manipulators 115 configured to manipulate multiple instruments 118. For example, the surgical robot 110 can include two, three, four, five, six, or more robotic manipulators 115, where each manipulator manipulates one or more corresponding instruments 118.

Each robotic manipulator 115 can include, for example, a robotic arm having a series of links connected by a series of joints. A distal end of the robotic arm can be configured to couple with the corresponding instrument, and a proximal end of the robotic arm can be supported by a base of the robot. Alternatively, or in combination, a robotic manipulator can include a carriage or motorized platform that may move along a track to control an instrument or interact with patient 114. In some instances, one or more users, such as one or more members of surgical staff 113, can mount or couple various instruments 118 to the various robotic manipulators 115 during initial set up and/or throughout a procedure to exchange instruments. As instruments are mounted to the various robotic manipulators 115, the robot 110 can be configured to detect presence and/or identify the corresponding instruments using sensing or identification technologies, such as optical sensing, magnetic sensing, radio frequency identification (RFID), or the like.

In the example shown in FIG. 1, surgical robot 110 is configured as a table-based system, where the robotic manipulators 115 are physically coupled to or integrated with a surgical table 116 (also referred to herein as an “operating table” or “patient support”), which supports patient 114. In some variations, the surgical robot 110 can be configured as a robotic cart that can be positioned beside the patient 114 and/or beside the surgical table 116. Alternatively, or in combination, the robot can be configured as a boom-based robot, where robotic manipulators descend from an overhead boom suspended above the patient, where such overhead boom is supported, for example, by a cart beside the patient or from a ceiling of an operating room. In some variations, the surgical robot 110 can include one or multiple robotic carts, where each cart supports one or multiple robotic manipulators or robotic arms, and where the multiple robotic carts are configured to operate in concert with each other. For example, in some variations, a distributed surgical robot can involve a modular cart system, where multiple carts are positioned beside the surgical table 116 and each cart supports a robotic manipulator 115 that manipulates a corresponding instrument 118.

Physician console 120 can be configured to provide inputs or receive outputs to or from the robot 110 or the instruments 118. As illustrated, physician console 120 includes one or more input devices 127, which a user (e.g., physician 123) can operate to provide commands for teleoperation of the robot 110. Input device 127 can include, for example, a handheld device that the physician 123 can manipulate with one or more hands to provide input to the system. In some variations, the physician console 120 can employ one or several types of input devices to provide various modes for the physician 123 to interact with the surgical system 100. Examples of input devices include pendants, gimbal-based controllers, graspers, touch sensors, trackballs, joysticks, buttons, and/or foot pedals.

Physician console 120 can also include one or more console displays, which can be configured to present images for observation by the physician 123. For example, a viewer 124 can be configured to display a scope view derived from endoscopic images (e.g., a video feed) captured by an instrument 118. This can facilitate control of the surgical robot 110 by the surgeon 123 via the input device(s) 127, while the surgeon views a real-time camera feed of the anatomical site within the patient's body. Alternatively, or in combination, the console display(s) such as viewer 124 can be configured to display supplemental information associated with the surgical system or procedure, such as, for example, pre-operative images, navigation information, interactive menus, and/or status information associated with the instruments, the robot, or the surgical system. Examples of displays that may be employed by surgical system include flat panel displays, stereoscopic displays, head-mounted displays, liquid crystal displays (LCD), organic light emitting diode (OLED) displays, touch screen displays, and/or various other types of electronic display devices.

The support tower 140 can interact with surgical robot 110, instruments 118, and/or physician console 120 to provide various supporting functionality to the system, such as vision processing, fluidics, and/or energy generation. For example, the support tower 140 can process images received from an endoscope, generate light to an endoscope to illuminate the surgical site, provide suction and/or irrigation from the surgical site, operate instrument tracking sensors such as shape sensors and/or electromagnetic (EM) sensors, and/or generate energy provided to one or more of the instruments 118 (e.g., for electrosurgery functions such as coagulating or cutting tissue). Alternatively, or in combination, support tower 140 can provide an interface for one or more users, such as surgical staff 113, to interact with the surgical system (e.g., provide inputs to the surgical system and/or observe outputs of the surgical system). In the illustrated example, support tower 140 includes one or more tower displays 142 that can be configured to present any of the same information described herein with respect to the physician console and/or additional information.

In the illustrated example, surgical robot 110, physician console 120, and support tower 140 are illustrated as separate components that may be positioned in various locations in procedure area 109. In some variations, any two or more of these components may be integral. For example, in some configurations, the support tower 140 may be provided as an integral component of the physician console 120 or surgical robot 110.

Control system 145 can be communicatively coupled to robot 110, physician console 120, and/or support tower 140. Control system 145 includes processing circuitry and memory configured to implement functions of surgical system 100, such as controlling or actuating robot 110, controlling or operating the instruments, or processing inputs or outputs to or from physician console 120. For example, processing circuitry of the control system 145 can be configured via hardware or software programming to implement any functions described further herein in connection with operation of surgical system 100, including carrying out any of the methods described herein. Examples of processing circuitry include one or more central processing units (CPUs), graphics processing units (GPUs), field programmable gate arrays (FPGAs), application specific integrated circuits (ASICs), or other processors configured to process inputs or outputs for the surgical system 100. As used herein, the term “processor” can encompass a single processing chip or integrated circuit, or multiple processing chips or integrated circuits that may be co-located or distributed in different locations and configured to execute functions described herein. Memory can store instructions that, when executed by the processor, cause the surgical system to perform any of the methods or functions described herein. As used herein, the term “memory” can encompass any suitable non-transitory computer readable medium embodied in one or several memory devices, such as hard drives, flash memory, solid state memory, storage discs, or tapes. Components of the control system 145 may be physically located in, or physically connected to, components of the surgical system 100, such as the robot 110, the physician console 120, and/or the support tower 140. Alternatively, or in combination components of control system 145 may be communicatively coupled to components of surgical system 100 via various wired or wireless interconnections.

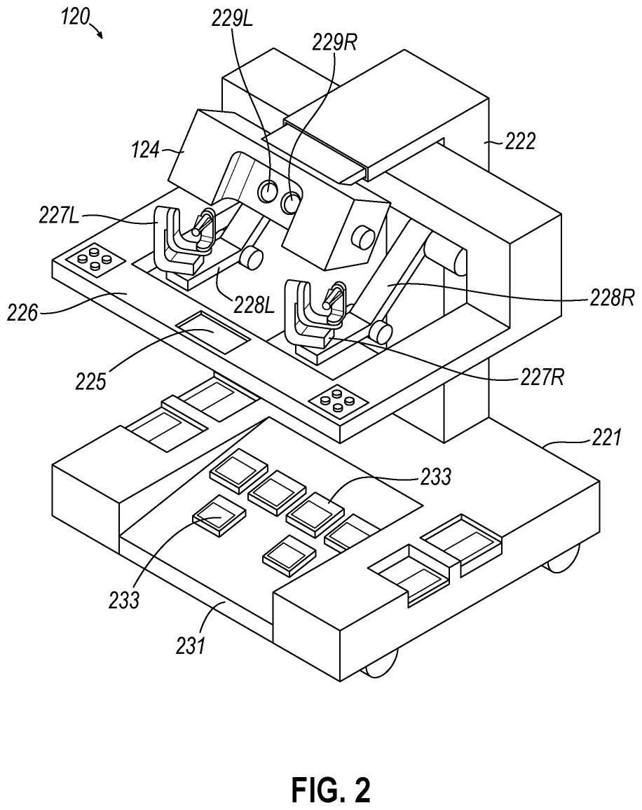

FIG. 2 depicts an example variation of physician console 120, in accordance with some embodiments. The physician console 120 can, for instance, be incorporated in surgical system 100 seen in FIG. 1. As noted above, physician console 120 (sometimes referred to herein as a “surgeon console”) can provide an interface for a user, such as a surgeon or physician, to interact with the surgical system 100. For example, a physician may interact with the physician console 120 to control the surgical robot and/or observe images of a surgical site.

As seen in FIG. 2, physician console 120 can include a console base 221, a pillar 222 (also referred to as a “column”) coupled to the console base 221, and a viewer 124 coupled to the pillar 222. Physician console 120 further includes an armrest 226, which is coupled to and supported by the pillar 222. Viewer 124 can be supported by the console base 221 via the pillar and can provide a primary display for a physician to view endoscopic images of the anatomical site. In some configurations, viewer 124 is configured to display anatomical images obtained from both flexible and rigid laparoscopes, as further described herein. In the illustrated example, viewer 124 is configured as an immersive, three-dimensional, stereoscopic display having a left eye display 229L and a right eye display 229R. This immersive display can present three-dimensional images to the physician when the physician inserts their head into the viewer housing. In some variations, the physician console 120 may be provided with an open design, where the viewer 124 is configured as a two-dimensional or three-dimensional flat panel display that can present endoscopic images to the physician without a need for the physician to insert their head into a viewer housing. In some variations, viewer 124 may be provided by a wearable headset (e.g., a head-mounted display).

In the example shown in FIG. 2, physician console 120 also includes a pair of input devices including a left hand input device (HID) 227L and a right HID 227R, configured to be manipulated by the physician's left and right hands, respectively (an HID is also sometimes referred to herein as a “human interface device”). Each of the HIDs can include a handle and/or finger inputs that are manipulated by a user's hands to control a corresponding instrument and/or corresponding robotic manipulator. For example, the left HID 227L may be controlled by a user's left hand to control a left-hand instrument manipulated by a first robotic arm of the surgical robot, and the right HID 227R may be controlled by a user's right hand to control a right-hand instrument manipulated by a second robotic arm of the surgical robot. In the illustrated example, each of the HIDs is physically supported by an armrest 226 of the physician console 120 and/or the pillar 222 by a respective positioning arm, including a left positioning arm 228L and a right positioning arm 228R. Such positioning arms can include a series of links and series of joints, including a gimbal-based support, that supports the respective HID in space while permitting the respective HID to be manipulated in six degrees of freedom to control a corresponding position (e.g., location and/or orientation) of the respective instrument. Alternatively, or in combination, each of the left HID or right HID can include graspers and/or buttons that may be actuated by the user's respective hands to actuate the instrument (e.g., to open or close instrument jaws) or control other functions of the surgical system. The illustrated configuration depicts grounded HIDs that are physically grounded to the console via positioning arms. In some variations, the physician console can employ ungrounded HIDs, such as free-floating and/or wireless input devices.

As seen in FIG. 2, physician console 120 can also include a foot pedal assembly 231 (e.g., a footboard) having one or more foot pedals 233. The foot pedal(s) 233 may be coupled to or otherwise positioned at the base 221 of the physician console and may be actuated by a user's feet to control various functionality of the system. For example, in some configurations, various foot pedals may be used to perform ancillary functions of the system, such as activating energy delivery, switching control of instruments, clutch instruments, firing a staple, or toggling a menu, for example.

In some variations, the physician console 120 can include one or more additional or secondary displays. In the illustrated example, physician console 120 includes an armrest display 225, which can be implemented as a touchscreen display positioned on the armrest 226. The armrest display 225 can provide an additional interface for a physician to interact with the system. For example, the armrest display 225 can provide an additional output interface for displaying various settings or status information associated with the surgical system 100. Alternatively, or in combination, the armrest display 225 can provide an input interface for controlling system settings.

In some variations, it may be beneficial for a surgical system to facilitate hybrid approaches or techniques, such as the use of both flexible and rigid instrumentation and/or access to internal anatomical sites via different types of entry points. For instance, a combined endoscopic and laparoscopic system may allow for complimentary hybrid techniques that combine benefits of both laparoscopy and endoscopy.

An example of a patient condition that may benefit from combined endoscopy and laparoscopic surgery is the diagnosis or treatment of colorectal polyps. Flexible endoscopic instrumentation may access the inside of a colon through a perineal access point (e.g., anus), then be advanced to a target polyp for further examination or treatment. If the polyp is benign or can be treated without a need for surgical resection, then the procedure may proceed using a purely endoscopic technique. In cases where further intervention is needed, the procedure may then escalate to laparoscopic intervention, where rigid instruments can perform surgical manipulation and/or resection of the malignant tissue. Alternatively, or in combination, such procedures may involve concomitant use of endoscopic and laparoscopic instrumentation to target the polyp or target site, for instance, to provide laparoscopic assistance to endoscopic examination or treatment of the polyp, and/or to provide endoscopic assistance to laparoscopic examination or treatment. Such hybrid techniques may benefit from the concomitant use of instruments for viewing or manipulating the same target anatomy from different perspectives, thereby facilitating enhanced surgical techniques or improved visualization of the target site.

Various types of patient conditions and procedures may benefit from hybrid techniques or combined endoscopic and laparoscopic surgery, providing potential for improved outcomes such as reduced complications, broadened use of organ preserving technique, reduced risk of injury, and reduced length of hospital stays. Examples of procedures that may benefit from combined endoscopic and laparoscopic surgery include upper GI procedures, lower GI procedures, and thoracic procedures.

Upper GI procedures can, for instance, involve one or more flexible instruments (such as an endoscope and/or working channel tools) introduced to an anatomical site (such as the stomach or upper GI tract) through a patient's mouth. With a combined endoscopic and laparoscopic system, one or more rigid instruments, such as a laparoscope and/or one or more other laparoscopic instruments, may access the anatomical site through one or more incisions on the patient's abdomen to access the same anatomical site from a different perspective (for example, from outside the stomach or upper GI tract).

Lower GI procedures can, for instance, involve one or more flexible instruments introduced to an anatomical site (such as the colon or lower GI tract) through a perineal access point. With a combined endoscopic and laparoscopic system, one or more rigid instruments may access the anatomical site through one or more incisions on the patient's abdomen to access the same anatomical site from a different perspective (for example, from outside the colon or lower GI tract).

Thoracic procedures can, for instance, involve one or more flexible instruments introduced to an anatomical site (such as the lung or pulmonary region) through a patient's mouth. With a combined endoscopic and laparoscopic system, one or more rigid instruments may access the anatomical site through one or more incisions on the patient's chest to access the same anatomical site from a different perspective (for example, from outside the lung or pulmonary region).

Robotic systems may facilitate these and other hybrid approaches, providing an integrated robotic platform that allows one or more users to control and/or visualize anatomy using endoscopic and laparoscopic approaches. In some variations, a single user may be able to control both endoscopic and instrumentation using a robotic surgical system.

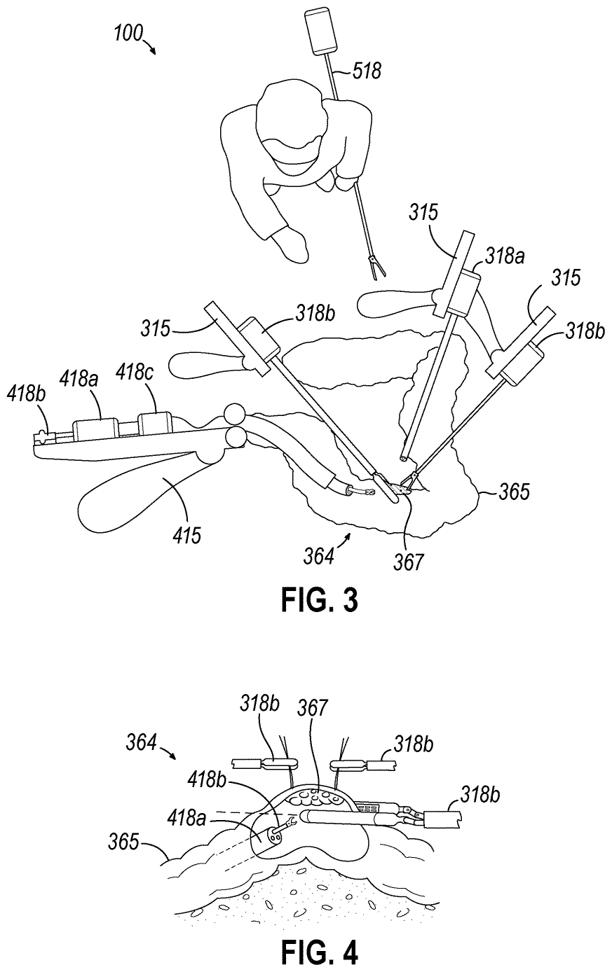

FIG. 3 depicts an example variation of surgical system 100 configured for combined endoscopic and laparoscopic surgery, in accordance with some embodiments.

As seen in FIG. 3, surgical system 100 employs multiple robotic manipulators 315, 415 and multiple instruments 315a-b, 415a-c interacting with an anatomical site 364. Here, anatomical site 364 encompasses an organ 365 (a colon in this example), where instruments operate on a target structure 367 within the organ (a polyp in this example) using both endoscopic (e.g., endoluminal) intervention and laparoscopic surgery.

In the illustrated example, three robotic manipulators 315 control three corresponding rigid instruments 318a-b from a laparoscopic approach, including a rigid scope 318a (e.g., a laparoscope) for capturing images of the anatomical site, and a pair of surgical instruments 318b (e.g., rigid laparoscopic instruments) for manipulating tissue. Rigid scope 318a can include a camera for capturing images of the anatomical site 364 while the surgical instruments 318b perform various tasks or manipulations on the anatomical site 364. For instance, rigid scope 318a may include one or more image sensors arranged at its distal tip, along with corresponding optics, a light source or light pipe at the distal tip for illuminating the anatomical site 364 while the surgical instruments 318b operate within the field of view (FOV) of the rigid scope 318a. In some variations, rigid scope 318a includes a stereoscopic camera to provide three-dimensional (3D) images. In some variations, rigid scope 318a includes a monoscopic camera to provide two-dimensional (2D) images. Each surgical instrument 318b can include an end effector adapted for a particular task, such as grasping, sealing, and the end effector may optionally be articulatable or actuatable by the corresponding robotic manipulator 315.

In the illustrated example, robotic manipulator 415 controls three flexible instruments 418a-c, which are mounted to the same robotic manipulator 415 in a coaxial or telescoping arrangement, and which interact with the anatomical site from an endoluminal approach. Here, the flexible instruments include a flexible scope 418a (e.g., a colonoscope), which extends through a channel of overtube 418c, and a working channel instrument 418b, which extends through a channel of the flexible scope 418a. Flexible scope 418a may be robotically steerable or controllable through the lumen (colon) to reach the target structure 367. Overtube 418c may also be robotically steerable or controllable to help support or guide the flexible scope 418a as it navigates through the lumen (colon). Working channel instrument 418b may be introduced through a working channel of the flexible scope 418a to interact with the target structure 367. For example, working channel instrument 418b may be manipulated to remove tissue, apply energy, or deliver therapeutics.

In some variations, one or more manual instruments may be utilized in concert with the surgical system 100. For example, a manual laparoscopic instrument 518 may be manipulated by a beside user through a laparoscopic port. Alternatively, or in combination, one or more of the illustrated instruments may be configured for manual control, such as the working channel instrument 418b or flexible scope 418a.

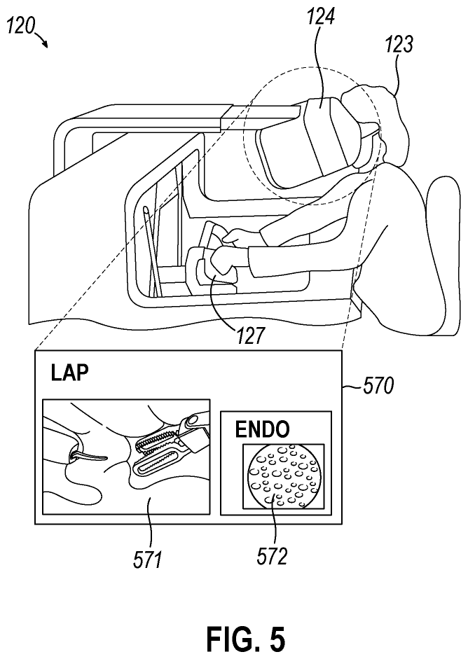

FIG. 4 depicts an enlarged view of the anatomical site 364, to further illustrate how endoluminal and laparoscopic instruments may be used in tandem to interact with operative site. Here, the distal tips of three surgical instruments 318b are shown manipulating the tissue (e.g., organ 365), while laparoscope 318a from FIG. 3 is not visible in this enlarged view. The distal tips of flexible endoscope 418a and working channel instrument 418b are shown observing or manipulating the target structure 367, while overtube 418c from FIG. 3 is not visible in this enlarged view. As illustrated, the endoluminal and laparoscopic instruments may interact with the anatomical site 364 in concert with each other from different approaches, such as opposing sides of the organ wall. Here, the flexible instruments 418a-b observe or interact with tissue from within the organ 365, while rigid instruments 318b interact with the target from outside the organ 365. Using these instruments in concert may allow various advanced techniques, such as, for example, grasping or positioning tissue with the laparoscopic instruments to facilitate visualization of the site with the endoluminal instruments.

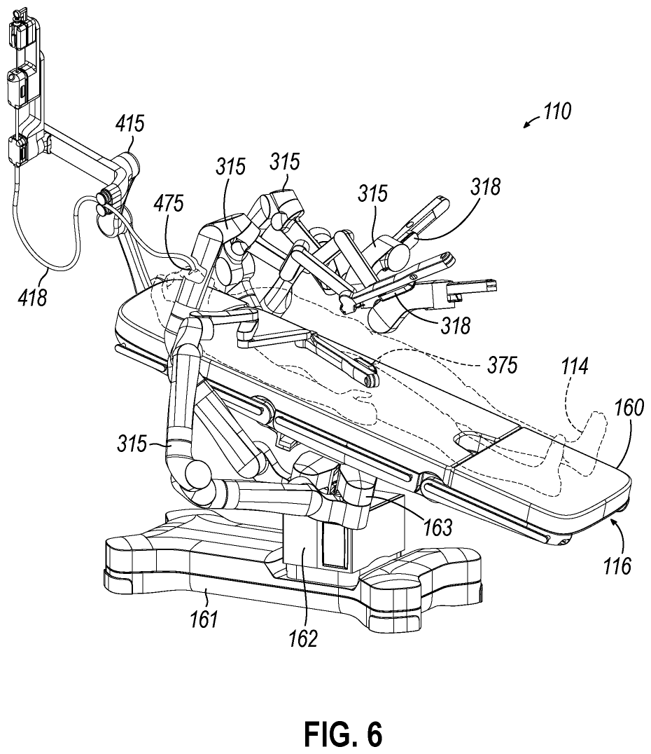

FIG. 5 depicts an example variation of physician console 120 configured for combined endoscopic and laparoscopic surgery, in accordance with some embodiments. Physician console 120 may be used, for instance, in connection with the surgical system 100 as seen in FIGS. 3-4.

As illustrated, physician console 120 may present a display a graphical interface 570 on viewer 124 for viewing by physician 123. Graphical interface 570 may display one or more endoscopic views or images of the anatomical site obtained from one or more scopes of the system. In the illustrated example, graphical interface 570 is configured to display both a laparoscopic view 571 and an endoluminal view 572. Laparoscopic view 571 can, for instance, include one or more images obtained from rigid scope 318a (FIG. 3). Endoluminal view 572 can, for instance, include one or more images obtained from flexible scope 418a (FIG. 3). Physician console 120 is configured to receive input at one or more input devices 127, including commands for controlling any one or more of the instruments while the physician observes the graphical interface 570 presented on the viewer.

In the illustrated example, graphical interface 570 presents both the laparoscopic view 571 and the endoluminal view 572 concurrently (simultaneously), which may allow physician to concurrently view the anatomical site captured by the scopes from different perspectives. The concurrently displayed views may be displayed, for instance, with a side-by-side configuration or a picture-in-picture configuration. In some variations, one of the views 571, 572 may be displayed larger than the other view or located more centrally within the display interface relative to the other view to be presented as a primary view, while the other of the views 571, 572 may be displayed smaller than the other view or less centrally than the other view to be presented as a secondary view. The physician console 120 may allow the user to select or switch between the views 571, 572 by providing input to one or more input devices, such as, for instance, one or more of the HIDs 227L, 227R, one or more of the foot pedals 233, and/or a touch interface of the armrest display 225 (FIG. 2). In some variations, views 571, 572 are displayed asynchronously, or not at the same time. In such variations, physician console 120 may be configured to receive input from physician 123 at any one or more of the input devices to switch between the views or select which of the views to be presented on the display.

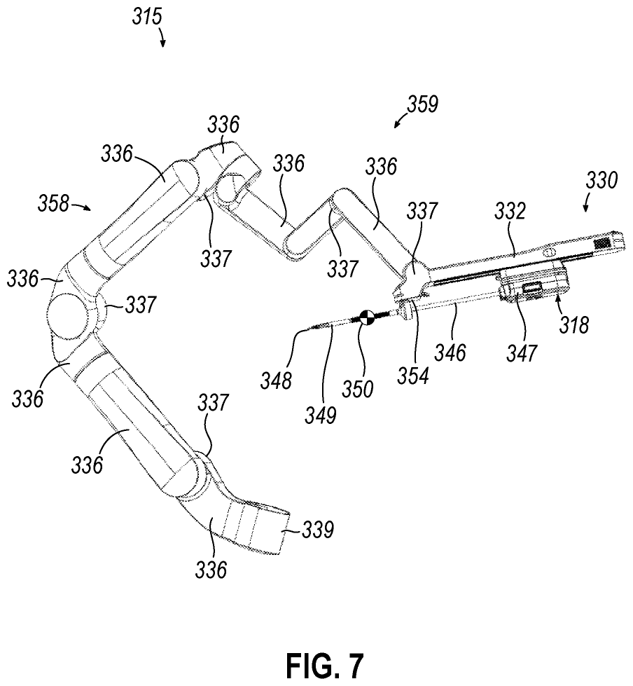

FIG. 6 depicts an example variation of surgical robot 110 configured for combined endoscopic and laparoscopic surgery, in accordance with some embodiments. Surgical robot 110 may be used, for instance, in connection with surgical system 100 as seen in FIGS. 3-4. Surgical robot 110 may be controlled, for instance, based on commands received from physician console 120 as seen in FIG. 5.

As seen in FIG. 6, surgical robot 110 includes multiple robotic manipulators 315, 415 coupled to operating table 116 (also referred to herein as a “surgical table”). Operating table 116 includes a table base 161, a column 162 extending vertically from the table base 161, and a table top 160 supported by the column 162. Patient 114 can be supported by the operating table 116 on the table top 160, which can be movable or actuatable relative to the table base 161 and/or column 162 to adjust positioning of the patient 114 supported thereon. For instance, the table top 160 may be adjustable to various positions, angles, or orientations relative to the table base 161 to provide a desired positioning of the patient for a given procedure or surgical task.

Robotic manipulators 315, 415 are mounted to a support 163, which can be coupled to the operating table 116 to provide a support base for each of the robotic manipulators 315, 415. In the illustrated example, support 163 is coupled to the table top 160 and may be fixed relative to the table top 160, such that the support 163 and the table top 160 move in concert with each other with unified motion as the table top 160 is actuated relative to the table base 161. In some variations, support 163 is coupled to the column 162, the table base 161, or a cart separate from the operating table 116, in which case the support 163 may be movable or actuatable independently from the table top 160. In the illustrated example, support 163 is positioned underneath the table top 160, such that the robotic manipulators 315, 415 can extend around edges of the table top 160 when in a deployed configuration to position the instruments 318, 418 in the workspace above or near the patient 114. In some variations, the robotic manipulators 315, 415 may be movable to a stowed configuration, where the robotic manipulators are positioned beneath the table top 160 for stowage.

Robotic manipulators 315, 415 can manipulate instruments 318, 418 that are introduced into the patient through anatomical openings 375, 475. In the illustrated example, surgical robot 110 includes four robotic manipulators 315 that hold and manipulate four corresponding rigid instruments 318. Each of the rigid instruments 318 may be introduced into the patient through a surgical incision 375 (e.g., a minimally invasive or laparoscopic incision). In some variations, each of the multiple rigid instruments 318 is introduced through its own respective port corresponding to its own respective incision. In some variations, two or more of the rigid instruments 318 are introduced through the same port corresponding to the same incision (e.g., for a single port surgical approach). In the illustrated example, surgical robot 110 also includes a robotic manipulator 418 configured to manipulate a flexible instrument, which has a flexible shaft that may be introduced into the patient through a natural orifice 475 (e.g., a mouth). In some variations, the robotic manipulators 315, 415 may be movable to various poses relative to the table top 160 to position the manipulators as appropriate for various procedures.

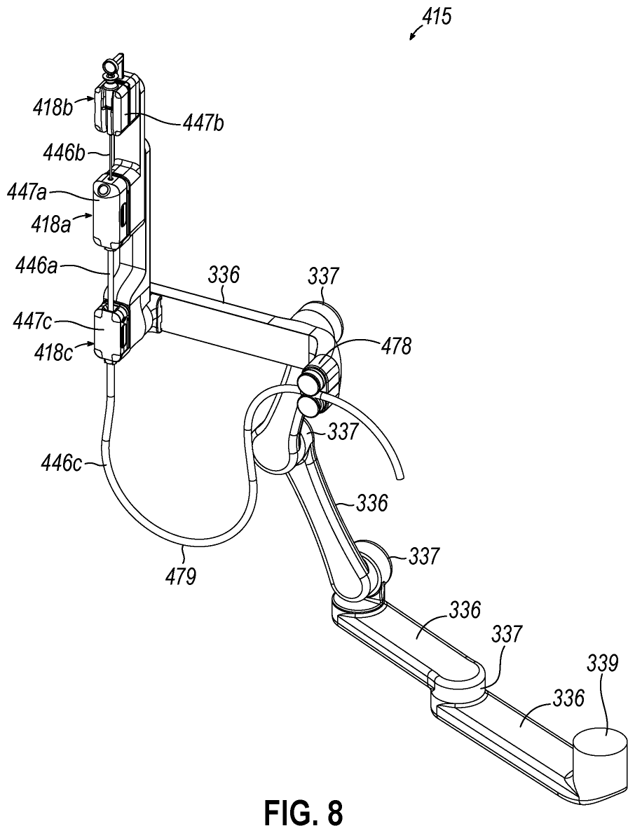

FIG. 7 depicts an example variation of robotic manipulator 315 for controlling a rigid instrument 318, in accordance with some embodiments. Robotic manipulator 315 may be used, for instance, in connection with surgical robot 110 as seen in FIG. 6 and/or surgical system 100 as seen in FIG. 3.

As seen in FIG. 7, robotic manipulator 315 includes a robotic arm having multiple links 336 connected by multiple joints 337. The links 336 may be arranged as a series of rigid bodies connected by the joints 337 to form a kinematic chain that terminates with rigid instrument 318. The robotic arm may include various types joints, such as one or more pitch joints, roll joints, and/or prismatic joints, each of which may constrain movement of its adjacent links around or along certain axes relative to others. Each joint 337 may include or be coupled to an actuator (e.g., a motor), which may be actuated to control movement of adjacent links relative to one another. Each joint 337 can include or be coupled to an encoder, which can measure position information associated with the joint (e.g., a joint angle), to provide robot kinematic data. A proximal end of the robotic arm may include an arm base 339, which may be coupled to, and supported by, a mounting structure of the surgical robot, such as support 163 (FIG. 6). For example, actuation of various joints of the robotic arm can move the distal end of the robotic arm to thereby control a position of the rigid instrument 318 in space, and to move the rigid instrument 318 relative to arm base 339.

A distal assembly 330 is arranged at the distal portion of the robotic arm. Distal assembly 330 includes a tool driver 332 (also referred to herein as an “instrument driver”) arranged at the distal end of the arm. Tool driver 332 is coupled to and supports rigid instrument 318. Tool driver 332 is also coupled to and supports cannula 349, which is configured to receive and guide rigid instrument 318.

The rigid instrument 318 includes an elongate shaft 346 and an instrument tip 348 arranged at a distal end of the elongate shaft 346. The elongate shaft 346 may rigidly support the instrument tip 348, which can provide an end effector for interacting with the anatomical site within the patient. Instrument shaft 346 can extend from the instrument base 347, which may provide a housing that contains mechanisms actuated by the tool driver 332 for actuating portions of the rigid instrument 318. In some variations, the instrument tip 348 includes a robotic wrist and jaws at the distal end of the tool, which can be actuated to manipulate tissue or perform surgical tasks. In some variations, the rigid instrument 318 is non-actuated, such as some variations of a rigid laparoscope. The plurality of the joints of the robotic arm can be actuated to position and orient the tool holder 332, thereby positioning and orienting the cannula 349 and/or the instrument tip 348.

A cannula interface 354 provides a cannula holding portion of the tool driver 332. Cannula interface 354 is configured to engage the cannula 349 via, for example, a clamp, latch, or mechanical attachment, to hold and stabilize the cannula 349 with respect to the tool driver and with respect to the rigid instrument 318 mounted to the tool driver. The tool driver can include a carriage that moves along an elongate track to thereby advance or retract the instrument shaft 346 through the cannula 349. The tool driver may be arranged at the distal end of a robotic arm such that articulation of the robotic arm positions and/or orients the tool holder 332 in space, thereby orienting the rigid instrument 318 and/or cannula 349.

FIG. 7 depicts an example of the robotic manipulator 315 adapted for a rigid surgical instrument (e.g., a laparoscopic instrument). As seen in FIG. 7, robotic manipulator 315 can manipulate or move rigid instrument 318 through a cannula 349 about a remote center of motion (RCM) 350, for example, by pivoting the rigid instrument 318 about RCM 350 in the direction of the arrow. As the elongate shaft 346 may rigidly support the instrument tip 348, moving or pivoting the instrument base 347 can cause a corresponding movement or pivoting of the instrument tip 348. The cannula 349 may, for example, provide a port that can be positioned at a small opening or incision on a patient's body, such as incision 375 (FIG. 6), to facilitate introduction of the rigid instrument 318 to an internal anatomical site. By maintaining the position of the RCM 350 during movements of the rigid instrument 318 and/or cannula 349, the robotic manipulator 318 can control a position of the instrument tip 348 of the rigid instrument 318 while avoiding undue trauma or stresses to the patient's body wall as that rigid instrument 318 is moved or manipulated.

In some variations, the plurality of links 336 and joints 337 of the robotic arm can be divided into two segments. The first segment 358 includes a proximal set of the links 336 and joints 337, and may be referred to as a setup arm because it can position and adjust the RCM in space relative to the mounting fixture. The second segment 359 can include a distal set of the links 336 and joints 337, and may be referred to as the spherical arm because it can move the surgical instrument within a generally spherical workspace. The second segment (spherical arm) 359 can include a mechanism that mechanically constrains movement of the tool driver 332 around RCM 350, and accordingly, constrains movement of the tools mounted to the tool driver 332 (including rigid instrument 318 and cannula 349), around RCM 350. Such a mechanism may be referred to as a mechanical RCM or mechanical-based RCM. For instance, a first one of the links 336 and a second one of the links 336 of the spherical arm can be operatively coupled with a pulley mechanism to form a parallelogram that mechanically constrains movement about the RCM. The spherical arm can, for instance, have at least two degrees of freedom (DOFs). The first segment (setup arm) 358 can, for instance, have at least five DOFs provided by five of the joints 337 in the first segment. The proximal end of the first segment 358 can be mounted to an arm support, while the distal end is coupled to the second segment 359.

In some variations, the robotic arm constrains motion about the RCM 350 via software or algorithms, rather than mechanical mechanisms. Such a configuration may be referred to as a software RCM or software-based RCM. In some variations, the robotic arm contains more or fewer joints and links, thereby providing more or fewer degrees of freedom for controlling motion of the robotic arm.

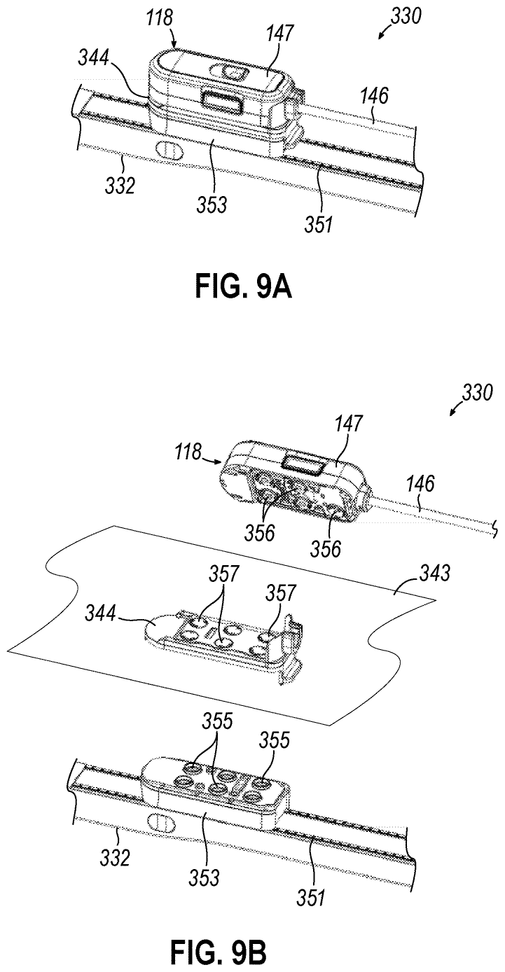

FIG. 8 depicts an example variation of robotic manipulator 415 for controlling one or more flexible instruments, in accordance with some embodiments. Robotic manipulator 415 may be used, for instance, in connection with surgical robot 110 as seen in FIG. 6 and/or surgical system 100 as seen in FIG. 3. Although robotic manipulator 415 may share features in common with robotic manipulator 315, here the robotic manipulator 415 has a different architecture adapted to manipulate flexible instruments rather than rigid instruments.

As seen in FIG. 8, robotic manipulator 415 includes a robotic arm having multiple links 336 connected by multiple joints 337. The links 336 may be arranged as a series of rigid bodies connected by the joints 337 to form a kinematic chain that terminates with distal assembly 330. A proximal end of the robotic arm may include an arm base 339, which may be coupled to, and supported by, a mounting structure of the surgical robot, such as support 163 (FIG. 6). For example, actuation of various joints of the robotic arm can move the distal end of the robotic arm to thereby control a position of the rigid instrument 318 in space, and to move the rigid instrument 318 relative to arm base 339.

A distal assembly 330 is arranged at the distal portion of the robotic arm. Distal assembly 330 includes a tool driver 332 (also referred to herein as an “instrument driver”) arranged at the distal end of the arm. In this example, tool driver 332 is coupled to and supports multiple flexible instruments 418a-c, which can include a flexible endoscope 418a, a working channel instrument 418b, and an overtube 418c. These flexible instruments 418a-c may be arranged in a coaxial or telescoping arrangement, and the tool driver 332 may include multiple instrument carriages arranged along a track to support the bases 447a-c of the respective instruments. Each of the instruments 418a-c includes a flexible shaft 446a-c that may extend from its corresponding instrument base 447a-c. Here, working channel instrument shaft 446b extends through the scope base 447a and into a working channel of the scope shaft 446a. Scope shaft 446a extends through the overtube base 447c and into a channel of the overtube shaft 446c. In some variations, the tool driver 332 may support more or fewer flexible instruments and/or different types of flexible instruments. For instance, the tool driver 332 may support one, two, three, or four flexible instruments in some variations. Each instrument base 447a-c may provide a housing that contains mechanisms actuated by the tool driver 332 for actuating portions of the corresponding flexible instrument 418a-c, for instance, to articulate, steer, or actuate the tip of the corresponding instrument. The plurality of the joints of the robotic arm can be actuated to position and orient the tool holder 332, thereby positioning and orienting the flexible instruments 418a-c.

FIG. 8 depicts an example of the robotic manipulator 415 adapted for a flexible instruments (e.g., endoluminal instruments). As seen in FIG. 8, tool driver 332 can be coupled to and support feedroller assembly 478, which is configured to receive one or more of the instrument shafts 446a-c. Feedroller assembly 478 can include one or more feedroller wheels (e.g., a pair of opposing rollers) that can engage with the instrument shaft(s) received therein, and can be driven to advance or retract the shaft. In the illustrated example, the flexible instruments shafts are shown with a service loop portion 446c, with the outermost overtube shaft 446c is visible, though it will be appreciated that the scope shaft 446a and working channel tool shaft 446b can be housed within the overtube shaft 446c. The service loop portion 479 provides slack in the instrument shaft(s). Feedroller assembly 478 is configured to take in the service loop portion 479 to advance the shaft(s), or let out to the service loop portion 479 to retract the shaft(s).

In some variations, the service loop portion and/or feedroller assembly 478 is omitted, in which case the robotic manipulator 415 can be configured to advance or retract the instrument shaft(s) solely via motion of the manipulator itself (e.g., articulation of the arm joints and/or movement of the instrument carriages of the tool driver 332). In some variations, an additional port, such as an introducer, may be coupled to or positioned proximate to the distal assembly 330 to facilite introducing the instrument shafts into the anatomical opening of the patient. In some variations, the robotic arm contains more or fewer joints and links, thereby providing more or fewer degrees of freedom for controlling motion of the robotic arm.

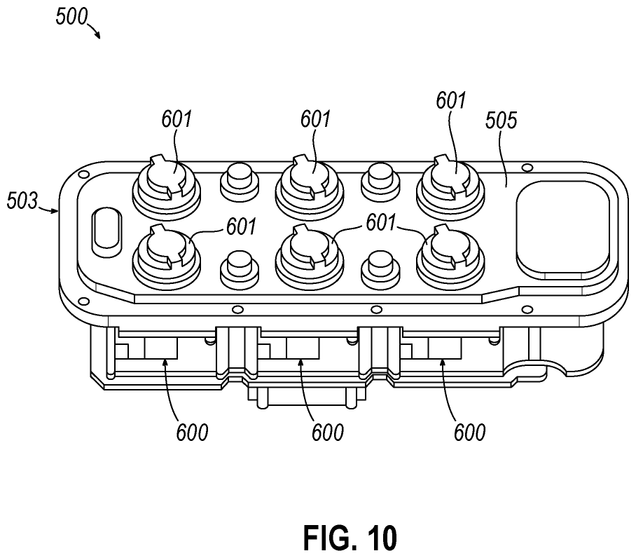

FIGS. 9A-9B depicts an example of a distal assembly 330 of a robotic manipulator, in accordance with some embodiments. FIG. 9A is an enlarged view of distal assembly 330 with instrument 118 coupled to tool driver 332. FIG. 9B depicts the distal assembly 330 in decoupled configuration, with the instrument 118 detached from tool driver 332 to illustrate various interfaces therebetween. Distal assembly 330 may be used in connection with any of the robotic manipulators described herein, including, for instance, robotic manipulators 315, 415 as seen in FIGS. 7-8. Instrument 118 may be configured in accordance with any of the instruments described herein, including, for instance, flexible and rigid instruments 315, 415

As illustrated, distal assembly 330 can include a tool driver 332 coupled with instrument 118 mounted thereon. The tool driver 332 may include an elongate track 351 (also referred to herein as a “stage”) having longitudinal guides, and a carriage 353, which is slidingly engaged with elongate track 351 and the longitudinal guides. The carriage 353 provides an instrument holding portion configured to receive an instrument base 147 of instrument 118. The carriage 353 can move along the elongate track 351 to thereby advance or retract the instrument 118.

Instrument base 147 may be coupled to the carriage 353 through an adapter 344. Adapter 344 can be coupled to a drape 343, such that the adapter 344 and drape 343 provide a barrier that separates the robotic manipulator (which may be capital equipment) from the instruments (which may be consumable equipment). Such a barrier may help maintain cleanliness for the robot or sterility within the field where instruments interact with the patient. The barrier or drape 343 may include portions that extend over the robotic manipulator, robotic arm, and/or portions of the surgical robot. In some variations, instrument base 147 can be coupled to the carriage 353 or tool driver 332 directly or without an adapter or barrier.

The tool driver 332 may actuate movements or functions of the instrument 118 mounted thereon. For example, tool driver 332 can actuate mechanisms in the housing of the instrument base 147 to actuate the instrument tip, such as through a cable system (e.g., pull wires) manipulated and controlled by actuated drives. The tool driver 332 may include different configurations of actuated drives. For example, as seen in FIG. 9B, the carriage 353 can include a set of drive outputs 355, which may engage a set of complementary drive inputs 356 on the instrument base 147 of surgical instrument 118. The drive outputs 355 may be configured to engage the drive inputs 356 on the instrument 118 through intervening drive couplers 357 on the adapter 344, allowing the adapter to maintain a barrier while transferring torque or actuation forces from the tool driver 332 to the instrument 118. The drive outputs 355 may include, for example, rotary discs, each coupled to a corresponding actuator that can include a motor (and optionally a gear transmission and/or encoder). The drive outputs 335, drive inputs 356, and drive couplers 357 may each include various engagement features to facilitate engagement and mating between the corresponding inputs, outputs, and couplers to facilitate torque or force transfer for actuation. For example, each of the drive inputs 355, drive outputs 356, and/or drive couplers 357 may include a set of teeth, dogs, or notches that complement each other and mate with each other so that, when engaged and actuated by the tool driver 332, the engaged set of inputs, output, and couplers move in unison. The drive outputs 355 may be arranged in any suitable manner. For example, as seen in FIG. 4B, the tool driver 332 may include six rotary drives 355 arranged in two rows, extending longitudinally along the instrument carriage 353. In some variations, the instrument carriage 353 includes more or fewer drive outputs, drive outputs positioned in different arrangements, and/or linear drive outputs instead of rotary discs.

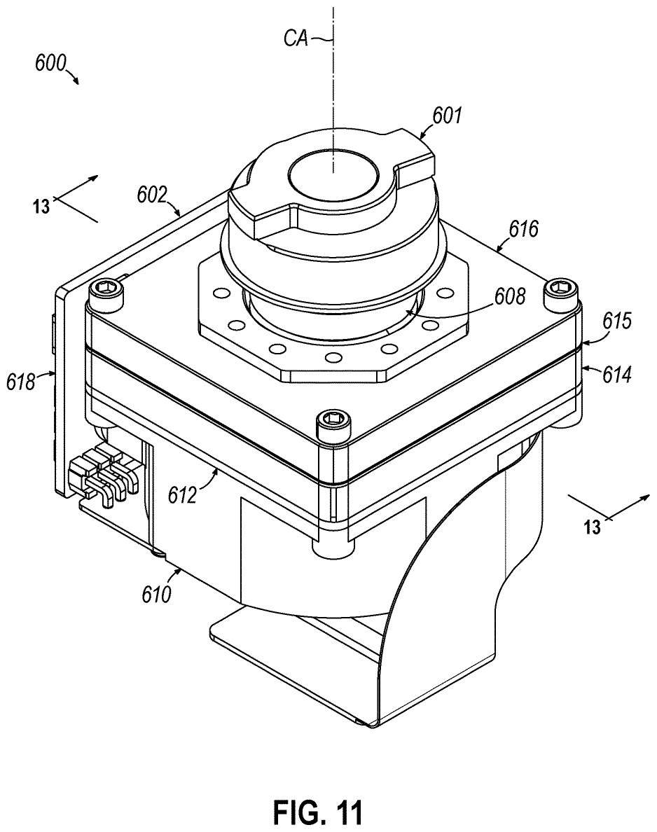

FIG. 10 depicts another example of a tool driver 500 that may be incorporated into distal assembly 330 in place of tool driver 332, in accordance with some embodiments. Tool driver 500 may be similar to tool driver 332 described above, except as otherwise described below. In this regard, tool driver 500 of the present example may include an elongate track (not shown) similar to elongate track 351 described above, and a carriage 503 similar to carriage 353 described above. In the example shown, carriage 503 includes a housing 505 and a plurality of actuators 600 housed within housing 505. Actuators 600 include corresponding drive outputs (also referred to as “output pucks”) 601 similar to drive outputs 355. Drive outputs 601 are each configured to rotate relative to housing 505 to thereby drive corresponding drive inputs (e.g., drive inputs 356). While drive output 601 is incorporated into actuator 600 in the example shown, it will be appreciated that drive output 601 may alternatively be considered a separate component of tool driver 500; and that actuator 600 may be coupled to drive output 601 in such cases.

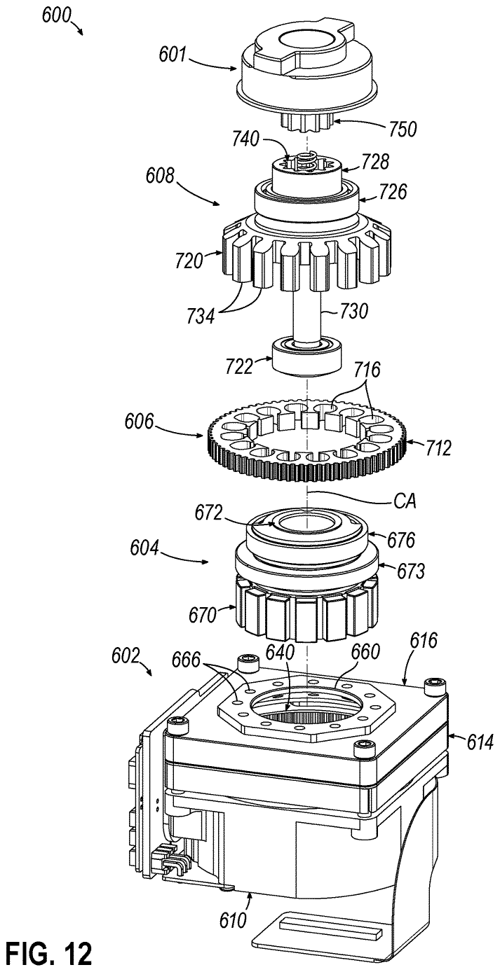

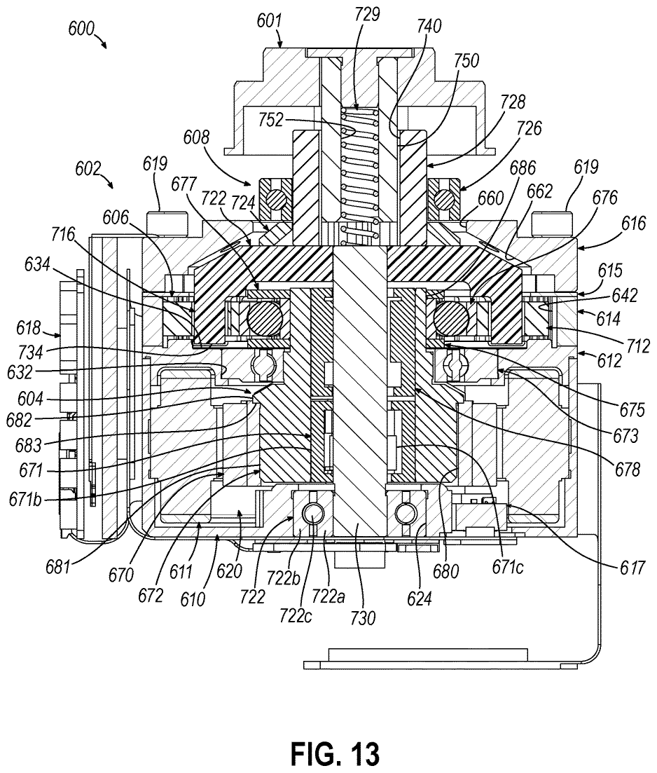

FIGS. 11-14B depict an example of an actuator 600 of a tool driver 500, in accordance with some embodiments.

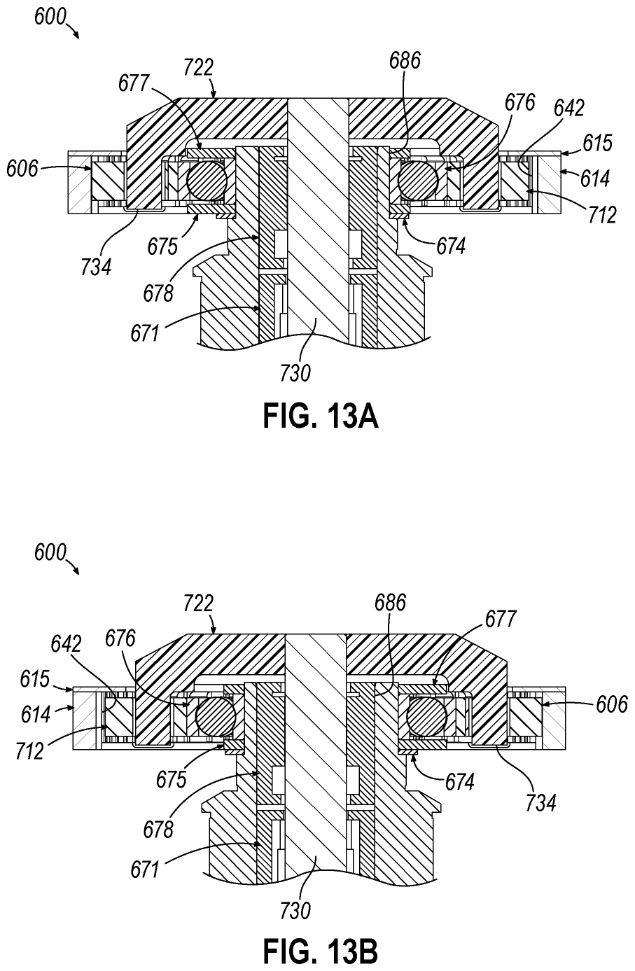

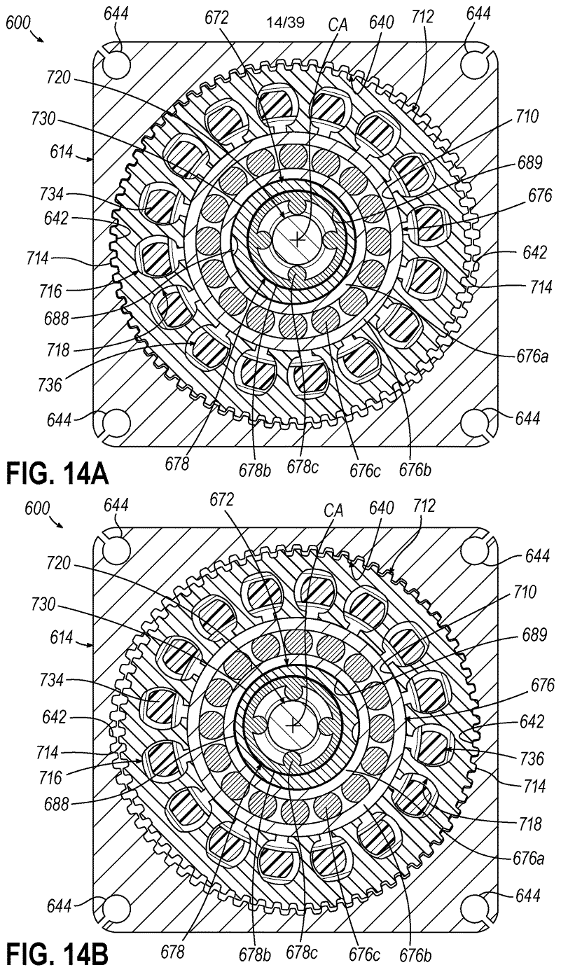

As illustrated, actuator 600 includes a housing assembly 602, a rotor assembly 604, a cycloid disc 606, an eccentric coupler assembly 608, and a drive output 601. Rotor assembly 604 is configured to drive eccentric movement of cycloid disc 606 relative to a central axis CA of rotor assembly 604; housing assembly 602 is configured to guide such eccentric movement of cycloid disc 606; and eccentric coupler assembly 608 is configured to convert such eccentric movement of cycloid disc 606 into rotation of drive output 601 about the central axis CA. For example, at least a portion of rotor assembly 604 may rotate about the central axis CA from a respective first rotational position in which cycloid disc 606 is offset toward a first side of the central axis CA such that at least a portion of eccentric coupler assembly 608 and drive output 601 are each in respective first rotational positions, as shown in FIGS. 13A and 14A, to a respective second rotational position in which cycloid disc 606 is offset toward a second side of the central axis CA such that at least a portion of eccentric coupler assembly 608 and drive output 601 are each in respective second rotational positions, as shown in FIGS. 13B and 14B. These and other functionalities of actuator 600 are described in greater detail below.

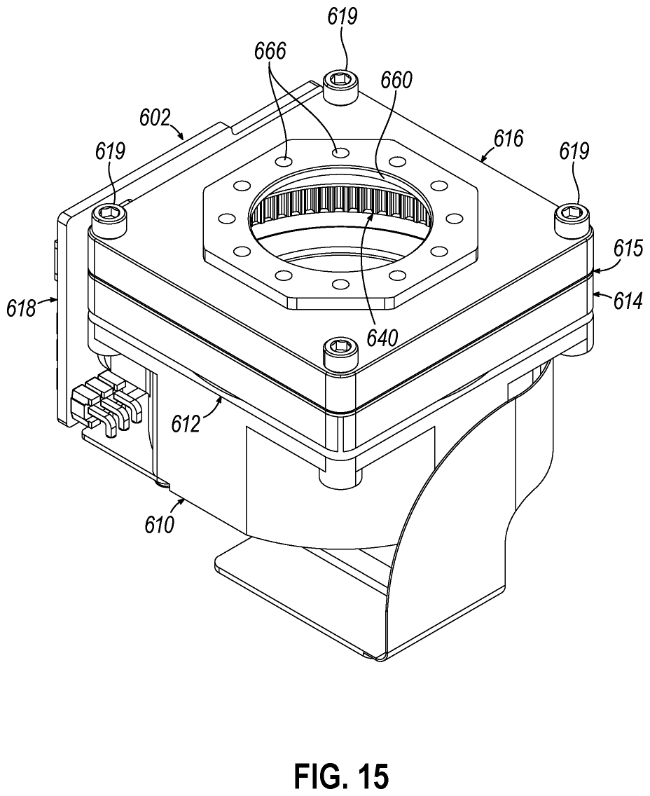

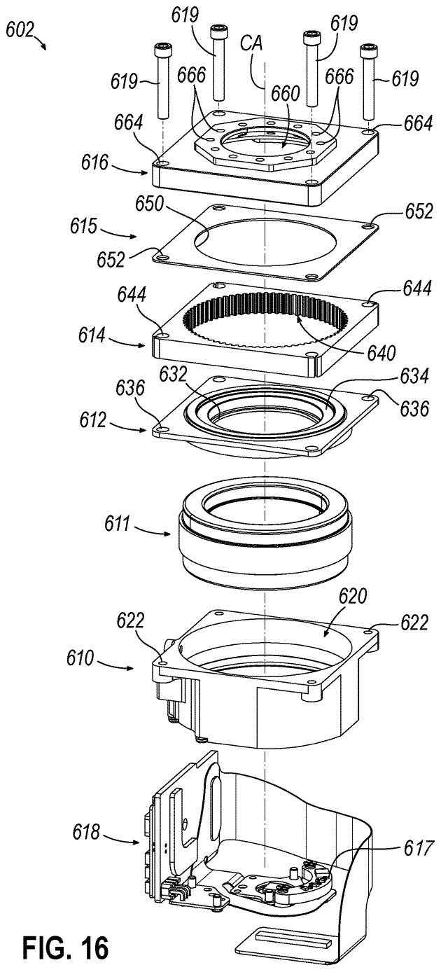

FIGS. 15-16 depict an example of a housing assembly 602 of actuator 600. In the example shown, housing assembly 602 includes a stator housing bottom 610, a stator 611, a stator housing top 612, a spline plate 614, a guard shim 615, a top cover 616, and a printed circuit assembly (“PCA”) 618. As illustrated, stator 611 is positioned within stator housing bottom 610 and captured therein via stator housing top 612, such that stator housing bottom 610 and stator housing top 612 collectively define a stator housing. Stator housing bottom 610, stator housing top 612, spline plate 614, guard shim 615, and top cover 616 are fixedly secured to each other in a stacked arrangement via a plurality of (e.g., four) fasteners in the form of bolts 619. More particularly, in the example shown, stator housing top 612 is disposed atop stator housing bottom 610; spline plate 614 is disposed atop stator housing top 612; guard shim 615 is disposed atop spline plate 614; and top cover 616 is disposed atop guard shim 615. PCA 618 may be fixedly secured to stator housing bottom 610 via one or more additional fasteners (not shown). In the example shown, PCA 618 includes an off-axis magnetic encoder 617 including a sensor array that is disposed inside a profile of stator 611 and configured to read corresponding magnets on rotor assembly 604. For example, magnetic encoder 617 may be disposed radially outwardly relative to the central axis CA and radially inwardly relative to stator 611.

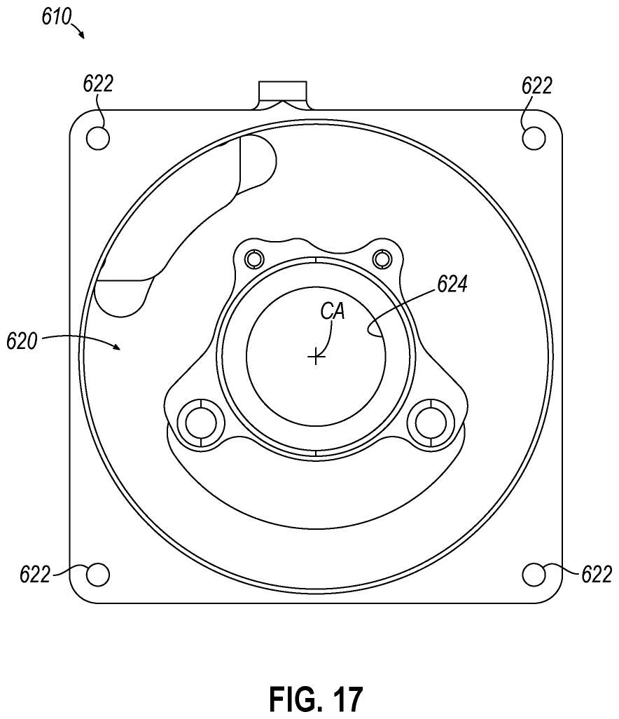

As shown in FIG. 17, stator housing bottom 610 of the present example includes a stator compartment 620 that is sized and configured to receive stator 611, which may be fixedly secured to stator housing bottom 610 via any suitable means. Stator housing bottom 610 of the example shown has a generally square-shaped profile (e.g., when viewed from above), and also includes a plurality of (e.g., four) threaded bores 622 positioned near an outer periphery (e.g., near respective corners) of stator housing bottom 610 and configured to threadably engage corresponding fasteners 619 for fixedly securing stator housing bottom 610 to stator housing top 612, spline plate 614, guard shim 615, and top cover 616. In the example shown, stator housing bottom 610 also includes a central lower sleeve portion 624 that is substantially centered relative to the central axis CA and that is configured to retain a portion of eccentric coupler assembly 608, as described in greater detail below.

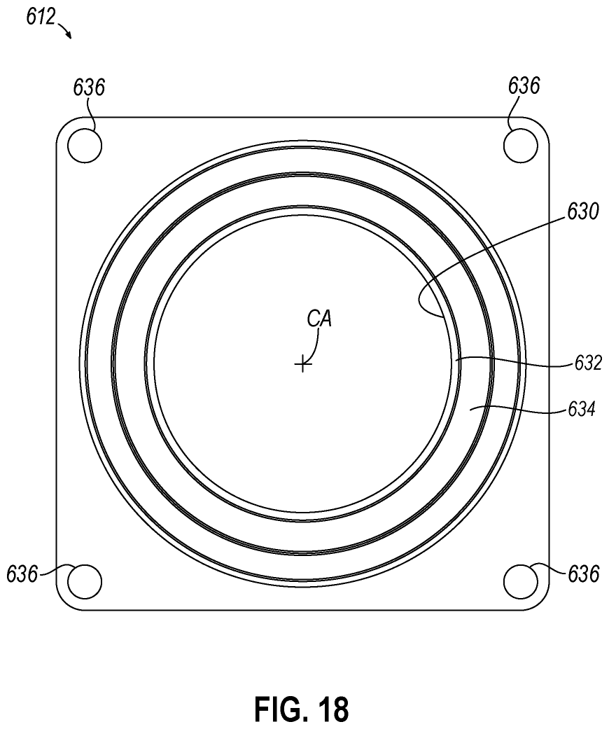

As shown in FIG. 18, stator housing top 612 of the present example includes a generally circular central bore 630 that is substantially centered relative to the central axis CA and that is sized and configured to permit at least a portion of rotor assembly 604 to pass therethrough; and further includes a generally annular, radially inner recess 632 and a generally annular, radially outer recess 634, the purposes of which are described below. Stator housing top 612 of the example shown has a generally square-shaped profile (e.g., when viewed from above), and also includes a plurality of (e.g., four) bores 636 positioned near an outer periphery (e.g., near respective corners) of stator housing top 612 and configured to receive corresponding fasteners 619 for fixedly securing stator housing top 612 to stator housing bottom 610, spline plate 614, guard shim 615, and top cover 616.

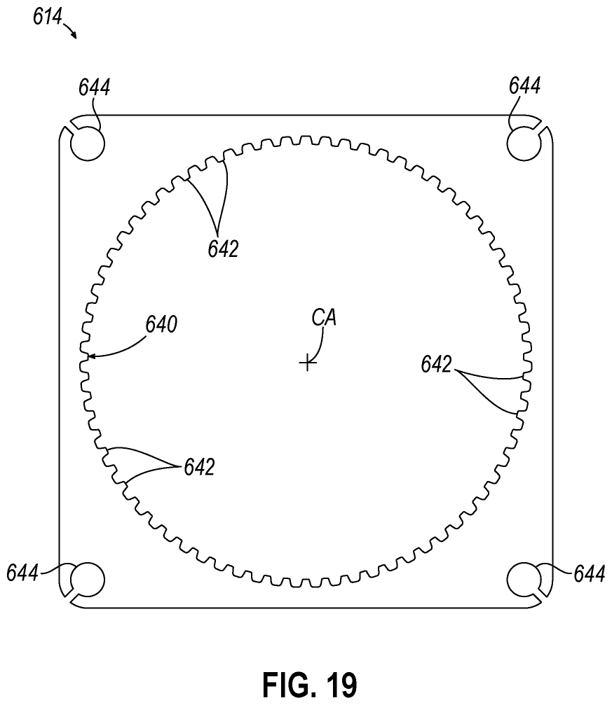

As shown in FIG. 19, spline plate 614 of the present example defines a generally circular internal ring gear 640 that is substantially centered relative to the central axis CA and that is configured to guide eccentric movement of cycloid disc 606 relative to the central axis CA. In the example shown, internal ring gear 640 has an involute geartooth profile. More particularly, internal ring gear 640 of the example shown includes a plurality of involute teeth 642 that are uniformly distributed around a circumference of internal ring gear 640 and that are configured to engage corresponding portions of cycloid disc 606, as described in greater detail below. Spline plate 614 of the example shown has a generally square-shaped profile (e.g., when viewed from above), and also includes a plurality of (e.g., four) bores 644 positioned near an outer periphery (e.g., near respective corners) of spline plate 614 and configured to receive corresponding fasteners 619 for fixedly securing spline plate 614 to stator housing bottom 610, stator housing top 612, guard shim 615, and top cover 616.

Guard shim 615 of the present example includes a generally circular central bore 650 that is substantially centered relative to the central axis CA and that is sized and configured to permit at least a portion of eccentric coupler assembly 608 to pass therethrough. Guard shim 615 may be configured to inhibit egress of lubricant from internal ring gear 640 of spline plate 614 toward top cover 616. Guard shim 615 of the example shown has a generally square-shaped profile (e.g., when viewed from above), and also includes a plurality of (e.g., four) bores 652 positioned near an outer periphery (e.g., near respective corners) of guard shim 615 and configured to receive corresponding fasteners 619 for fixedly securing guard shim 615 to stator housing bottom 610, stator housing top 612, spline plate 614, and top cover 616.

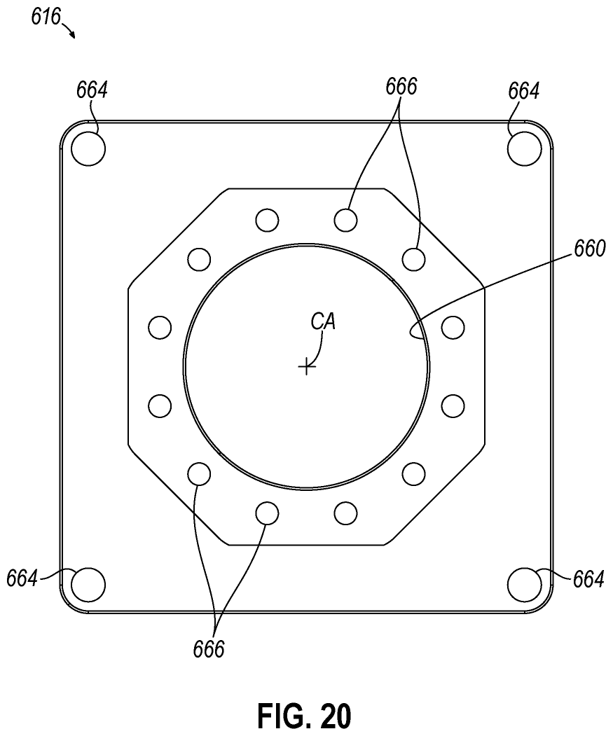

As shown in FIG. 20, top cover 616 of the present example includes a generally circular central bore 660 that is substantially centered relative to the central axis CA and that is sized and configured to permit at least a portion of eccentric coupler assembly 608 to pass therethrough. Top cover 616 of the present example also includes a generally frustoconical recess 662 that is provided in a lower surface of top cover 616 radially outwardly relative to central bore 660 and that is sized and configured to accommodate at least a portion of eccentric coupler assembly 608. Top cover 616 of the example shown has a generally square-shaped profile (e.g., when viewed from above), and also includes a plurality of (e.g., four) bores 664 positioned near an outer periphery (e.g., near respective corners) of top cover 616 and configured to receive corresponding fasteners 619 for fixedly securing top cover 616 to stator housing bottom 610, stator housing top 612, spline plate 614, and guard shim 615. In the example shown, top cover 616 also includes a generally circular array of threaded bores 666 positioned radially outwardly relative to central bore 660 and configured to threadably engage additional fasteners (not shown) for fixedly securing top cover 616 (and thus housing assembly 602) to another portion of the tool driver 500, such as to housing 505.

In some versions, top cover 616 may include a plurality of thin shear regions (not shown), which may be positioned near respective corners of top cover 616 (e.g., radially inwardly of respective bores 664). Strain gauges (not shown) may be disposed on such thin shear regions to facilitate monitoring of torque within actuator 600. Such strain gauges may be configured to generate one or more signals indicative of the amount of strain on their respective thin shear regions, and to send such signals to control system 145. For example, the strain gauges may be communicatively coupled to control system 145, such as via PCA 618. Processing circuitry of the control system 145 may be configured to determine the amounts of strain on the thin shear regions based on the signals received from the strain gauges; and to correlate such strain to the torque being applied between radially inner and radially outer portions of top cover 616. Thus, control system 145 may accurately determine the torque being applied between the radially outer portion of top cover 616 (e.g., together with stator housing bottom 610, stator housing top 612, spline plate 614, and guard shim 615) and the radially inner portion of top cover 616 (e.g., together with housing 505), based on the signals received from the strain gauges.

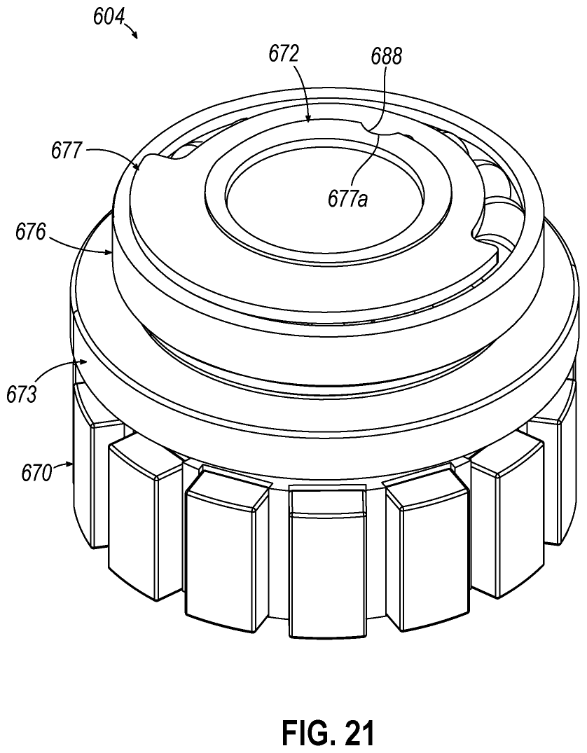

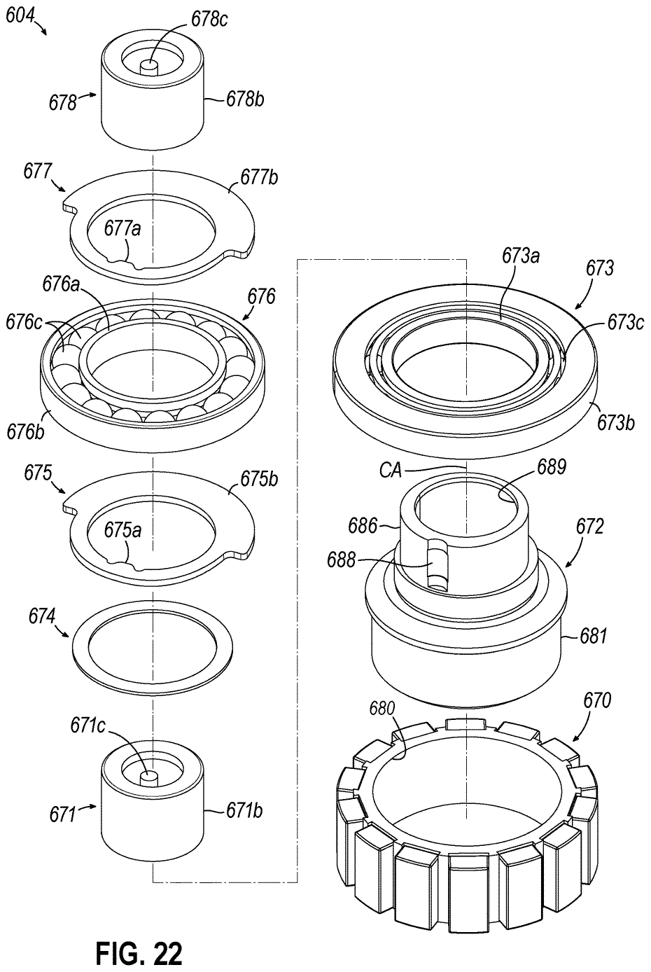

FIGS. 21-22 depict an example of a rotor assembly 604 of actuator 600. In the example shown, rotor assembly 604 includes a rotor 670, a lower needle bearing 671, a rotor shaft 672, a lower ball bearing 673, a balancing shim 674, a lower balancing disc 675, an upper ball bearing 676, an upper balancing disk 677, and an upper needle bearing 678. Rotor 670 is surrounded by stator 611 and configured to be selectively rotated thereby about the central axis CA, such that stator 611 and rotor 670 cooperate with each other to at least partially define an electric motor. Magnets of rotor 670 may be disposed above magnetic encoder 617, such that the magnets of rotor 670 may each be selectively read by magnetic encoder 617 to facilitate monitoring of an angular position of rotor 670 about the central axis CA. As illustrated, rotor shaft 672 is fixedly secured to rotor 670 such that rotor shaft 672 is configured to rotate together with rotor 670 about the central axis CA. More particularly, in the example shown, rotor 670 includes a generally circular central bore 680 that is substantially centered relative to the central axis CA and that is sized and configured to securely receive at least a portion of rotor shaft 672. For example, central bore 680 may be sized and configured to provide a friction fit with a portion of rotor shaft 672.

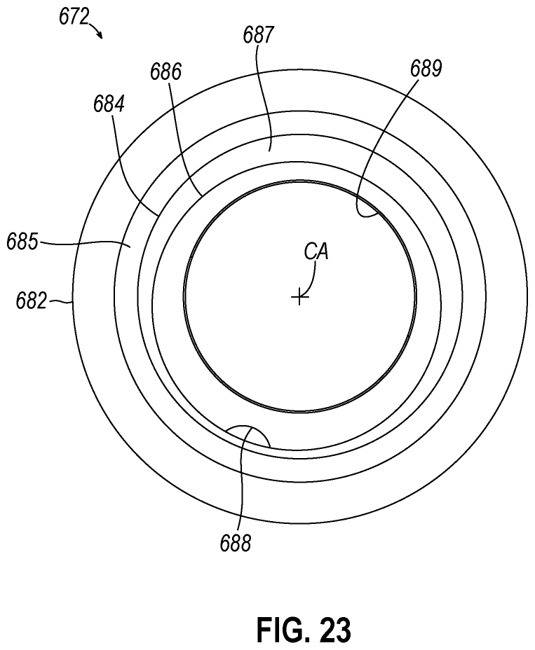

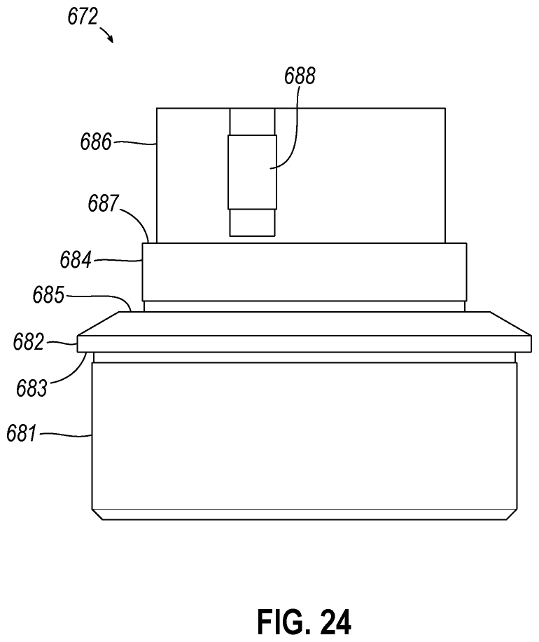

As shown in FIGS. 23-24, rotor shaft 672 of the present example includes a generally cylindrical input portion 681 that is substantially centered relative to the central axis CA and that is sized and configured to be securely received within central bore 680 of rotor 670. For example, input portion 681 may be sized and configured to provide a friction fit with central bore 680 of rotor 670. Rotor shaft 672 of the present example also includes a tapered, generally annular flange 682 extending radially outwardly relative to an upper end of input portion 681 to thereby define a generally annular, downwardly-facing shoulder 683 that is sized and configured to abut an upper surface of rotor 670. In the example shown, rotor shaft 672 also includes a generally cylindrical hub 684 extending upwardly relative to input portion 681 and positioned radially inwardly relative to flange 682 to thereby define a generally annular, upwardly-facing ledge 685 that is sized and configured to support at least a portion of lower ball bearing 673. In this regard, hub 684 of the present example is sized and configured to be securely received by at least a portion of lower ball bearing 673, as described in greater detail below.

Rotor shaft 672 of the present example also includes a generally cylindrical output portion 686 that is substantially eccentric relative to the central axis CA and that is sized and configured to be securely received by at least a portion of upper ball bearing 676, as described in greater detail below. In the example shown, output portion 686 is positioned radially inwardly relative to hub 684 to thereby define a generally annular, upwardly-facing ledge 687 (e.g., having a varying width) that is sized and configured to support at least a portion of balancing shim 674. As shown, a groove 688 extends vertically along a radially outer surface of output portion 686. More particularly, groove 688 extends vertically along a portion of the radially outer surface of output portion 686 that is farthest away from the central axis CA. Groove 688 is sized and configured to receive corresponding portions of balancing discs 675, 677, as described in greater detail below. Rotor shaft 672 of the present example also includes a generally cylindrical central bore 689 that is substantially centered relative to the central axis CA and that is sized and configured to permit at least a portion of eccentric coupler assembly 608 to pass therethrough. More particularly, central bore 689 is sized and configured to receive lower needle bearing 671 and upper needle bearing 678, which are, in turn, sized and configured to receive corresponding portions of eccentric coupler assembly 608, as described in greater detail below.

Lower ball bearing 673 of the present example includes an inner race 673a, an outer race 673b, and a plurality of bearing balls 673c disposed therebetween. In the example shown, inner race 673a is sized and configured to securely receive hub 684 and to rest upon ledge 685 of rotor shaft 672 such that inner race 673a may rotate together with hub 684; while outer race 673b is sized and configured to be securely received by inner recess 632 of stator housing top 612 such that outer race 673b may remain static relative to stator housing top 612. Thus, lower ball bearing 673 may be substantially centered relative to the central axis CA, and may minimize rolling resistance between rotor shaft 672 and housing assembly 602.

Upper ball bearing 676 of the present example includes an inner race 676a, an outer race 676b, and a plurality of bearing balls 676c disposed therebetween. In the example shown, inner race 676a is sized and configured to securely receive output portion 686 of rotor shaft 672 such that inner race 676a may rotate together with output portion 686; while outer race 676b is sized and configured to be securely received by corresponding portions of cycloid disc 606 such that outer race 676b may move together with cycloid disc 606, as described in greater detail below. Thus, upper ball bearing 673 may be substantially eccentric relative to the central axis CA, and may minimize rolling resistance between rotor shaft 672 and cycloid disc 606.

Lower needle bearing 671 and upper needle bearing 678 of the present example each include a respective outer race 671b, 678b, and a respective plurality of substantially cylindrical bearing rollers 671c, 678c. While not shown, needle bearings 671, 678 may each include a respective inner race sized and configured to securely receive corresponding portions of eccentric coupler assembly 608 such that they may rotate together with such portions of eccentric coupler assembly 608; while outer races 671b, 678b are each sized and configured to be securely received within central bore 689 of rotor shaft 672 such that outer races 671b, 678b may rotate together with rotor shaft 672. Thus, lower needle bearing 671 and upper needle bearing 678 may each be substantially centered relative to the central axis CA, and may minimize rolling resistance between rotor shaft 672 and eccentric coupler assembly 608.

While rotor assembly 604 of the present example includes lower needle bearing 671, lower ball bearing 673, upper ball bearing 676, and upper needle bearing 678, it will be appreciated that rotor assembly 604 may include any other suitable type(s) and/or quantity of bearings. For example, any other suitable type of rolling-element bearing(s) may be used in place of any one or more of lower needle bearing 671, lower ball bearing 673, upper ball bearing 676, and/or upper needle bearing 678.

Balancing shim 674 of the present example is sized and configured to securely receive output portion 686 and to rest upon ledge 687 of rotor shaft 672 and/or upon inner race 673a of lower ball bearing 673 such that balancing shim 674 may rotate together with output portion 686. In the example shown, balancing shim 674 is disposed below lower balancing disc 675.

Lower balancing disc 675 and upper balancing disc 677 of the present example are each sized and configured to securely receive output portion 686 of rotor shaft 672 such that lower balancing disc 675 and upper balancing disc 677 may rotate together with output portion 686. In the example shown, upper ball bearing 673 is sandwiched between lower balancing disc 675 and upper balancing disc 677. Lower balancing disc 675 and upper balancing disc 677 of the present example each include a radially inwardly-extending tongue 675a, 677a sized and configured to be securely received within groove 688 of rotor shaft 672; and a radially outwardly-extending arch 675b, 677b positioned generally opposite the respective tongue 675a, 677a. In this manner, lower balancing disc 675 and upper balancing disc 677 may be configured to balance cycloid disc 606 and thereby reduce or eliminate vibrations that might otherwise be generated by cycloid disc 606.

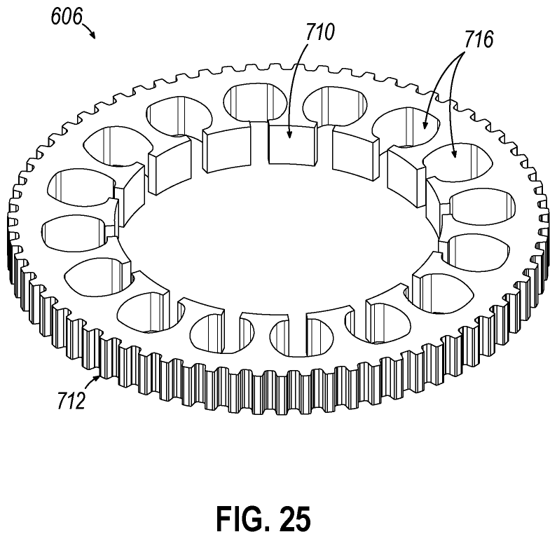

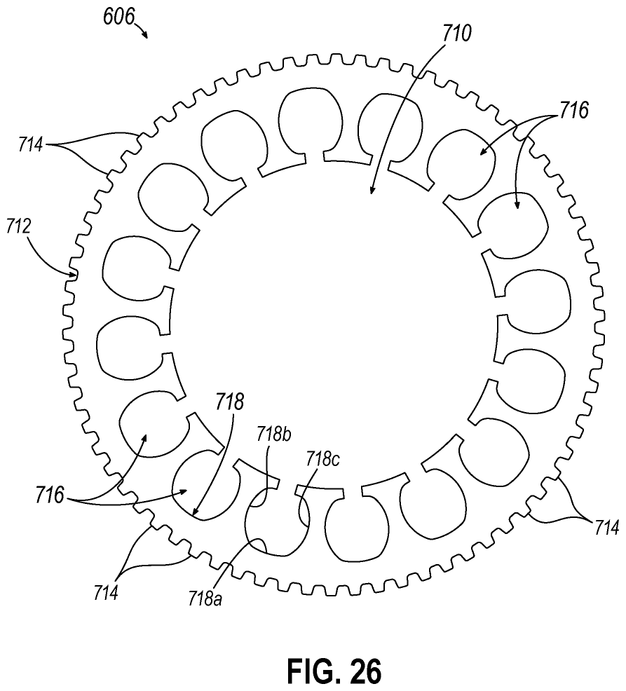

FIGS. 25-26 depict an example of a cycloid disc 606 of actuator 600. In the example shown, cycloid disc 606 includes a generally circular central bore 710 that is sized and configured to securely receive outer race 673b of the eccentrically-mounted upper ball bearing 673. For example, central bore 710 may be sized and configured to provide a friction fit with outer race 673b. Cycloid disc 606 of the present example also defines a generally circular external ring gear 712 that is configured to engage internal ring gear 640 of spline plate 614, such that internal ring gear 640 may guide eccentric movement of cycloid disc 606 relative to the central axis CA. In the example shown, external ring gear 712 has an involute geartooth profile. More particularly, external ring gear 712 of the example shown includes a plurality of involute teeth 714 that are uniformly distributed around a circumference of external ring gear 712 and that are configured to engage involute teeth 642 of internal ring gear 640. The involute profiles of teeth 642, 714 may provide ring gears 640, 712 with a consistent pressure angle. In some versions, the relative movement between cycloid disc 606 and the spline plate 614 may be constrained to ensure that at least a minimum threshold number of (e.g., five) involute teeth 714 of cycloid disc 606 are engaged with corresponding involute teeth 642 of spline plate 614 during operation of actuator 600. In this regard, maintaining continuous contact across such a predetermined threshold number of teeth 642, 714 may provide actuator 600 with a substantially high load carrying capacity. Thus, in response to rotation of rotor shaft 672, the eccentrically-mounted upper ball bearing 673 may drive cycloid disc 606 in an eccentric, generally cycloidal motion; such that cycloid disc 606 may oscillate (e.g., radially) at a first speed (e.g., the rotational speed of rotor shaft 672), and may rotate at a second speed less than the first speed.

Cycloid disc 606 of the present example also includes a plurality of (e.g., 16) receptacles 716 defined by corresponding bushing surfaces 718. In the example shown, receptacles 716 are uniformly distributed about central bore 710 in a generally circular array, and are sized and configured to receive corresponding portions of eccentric coupler assembly 608, as described in greater detail below. Receptacles 716 of the present example are each generally C-shaped (e.g., when viewed from above), with each bushing surface 718 having a generally flat radially-outer portion 718a and a pair of generally concave side portions 718b, 718c extending radially inwardly from respective ends of the corresponding radially-outer portion 718a. While receptacles 716 of the present example each open into central bore 710, receptacles 716 may alternatively each be closed off from central bore 710.

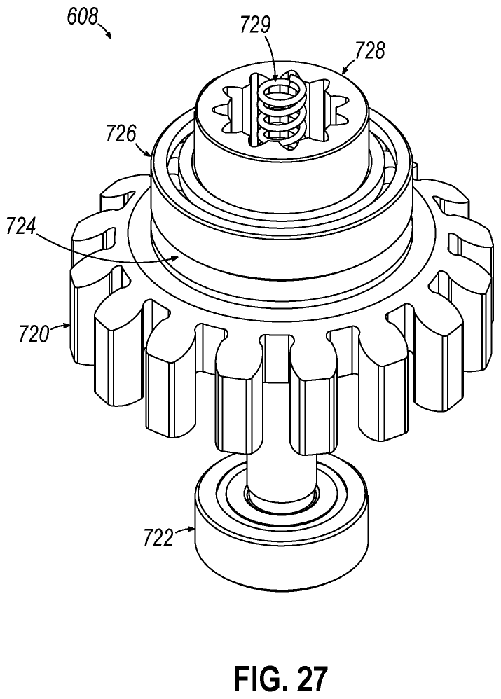

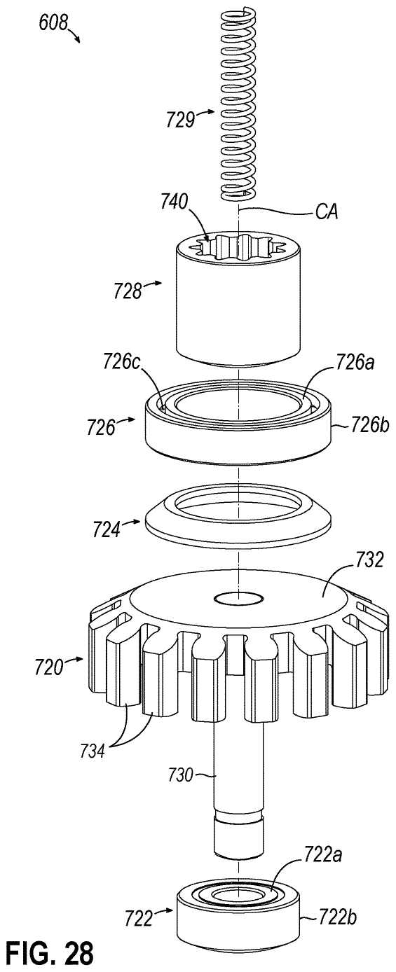

FIGS. 27-28 depict an example of an eccentric coupler assembly 608. In the example shown, eccentric coupler assembly 608 includes an eccentric coupler input 720, a lower ball bearing 722, a spacer 724, an upper ball bearing 726, an eccentric coupler output 728, and a resilient biasing member in the form of a compression spring 729.

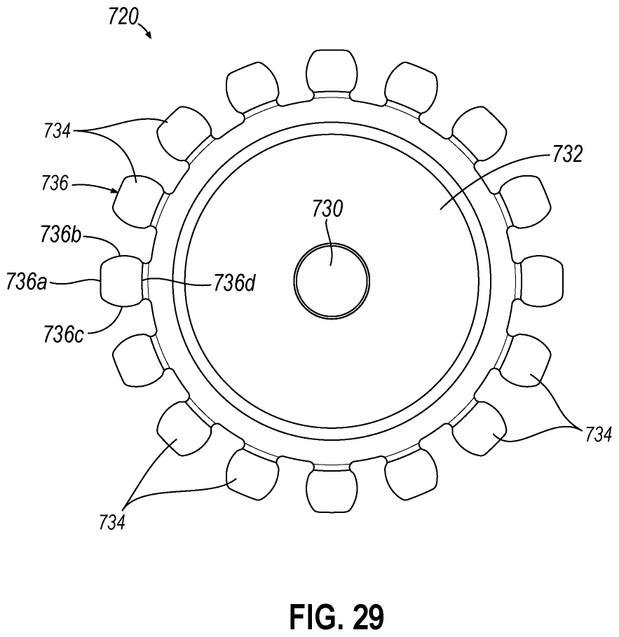

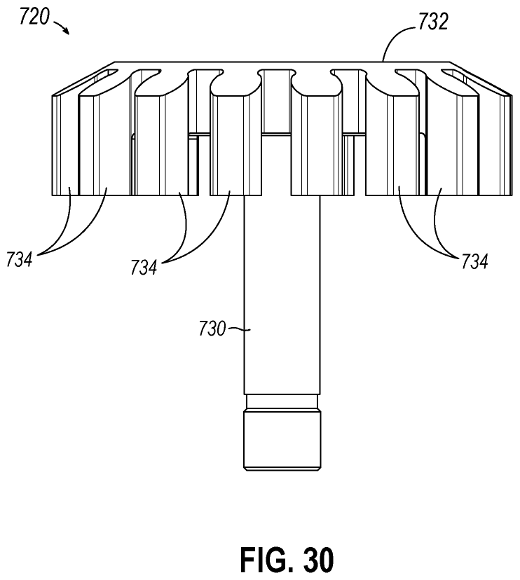

As shown in FIGS. 29-30, eccentric coupler input 720 of the present example includes a generally cylindrical stem 730, a generally circular head 732 extending radially outwardly from an upper end of stem 730, and a plurality of (e.g., 16) pins 734 extending downwardly from an outer periphery of head 732 and defining corresponding bushing surfaces 736. Stem 730 may be sized and configured to be received within inner races of needle bearings 671, 678 such that the inner races of needle bearings 671, 678 may rotate together with stem 730 relative to rotor shaft 672. In the example shown, pins 734 are uniformly distributed around a circumference of head 732 in a generally circular array, and are sized and configured to be received within corresponding receptacles 716 of cycloid disc 606. Pins 734 may extend through the corresponding receptacles 716, such that bottom surfaces of pins 734 may confront radially outer recess 634 of stator housing top 612. In some versions, a first thrust bushing (not shown) may be securely received within outer recess 634, and/or a second thrust bushing (not shown) may be fixedly secured to the bottom surfaces of pins 734 for reducing friction between eccentric coupler input 720 and stator housing top 612. Pins 734 of the present example are each generally D-shaped (e.g., when viewed from below), with each bushing surface 736 having a generally flat radially-outer portion 736a and a pair of generally convex side portions 736b, 736c extending radially inwardly from respective ends of the corresponding radially-outer portion 736a to a respective generally flat radially-inner portion 736d. Pins 734 are each sized and shaped relative to the corresponding receptacles 716 to permit relative radial and/or circumferential movement between each pin 734 and the corresponding receptacle 716 when pins 734 are received within the corresponding receptacles 716; to thereby permit each bushing surface 718 of cycloid disc 606 to selectively (e.g., intermittently) engage with the corresponding bushing surface 736 of eccentric coupler input 720. For example, receptacles 716 may be larger than pins 734 by an amount substantially equal to the eccentricity of output portion 686 of rotor shaft 672. In some versions, the relative movement between each pin 734 and the corresponding receptacle 716 may be constrained to ensure that at least a minimum threshold number of (e.g., three) bushing surfaces 718 of cycloid disc 606 are engaged with the corresponding bushing surfaces 736 of eccentric coupler input 720 during operation of actuator 600. In this regard, maintaining continuous contact across such a predetermined threshold number of bushing surfaces 718, 736 may provide actuator 600 with a substantially high load carrying capacity. Thus, in response to rotation of cycloid disc 606 at the second speed, bushing surfaces 718 of cycloid disc 606 may transmit such rotation directly to eccentric coupler input 720 via the corresponding bushing surfaces 736 so that eccentric coupler input 720 may also rotate at the second speed (e.g., without transmitting any radial motion of cycloid disc 606 to eccentric coupler input 720).

Spacer 724 of the present example is generally frustoconical, and is sized and configured to accommodate at least a portion of eccentric coupler output 728 and to be rotatably received within central bore 660 of top cover 616. In the example shown, spacer 724 rests upon head 732 of eccentric coupler input 720.

Eccentric coupler output 728 of the present example is fixedly secured to head 732 of eccentric coupler input 720 such that eccentric coupler output 728 is configured to rotate together with eccentric coupler input 720. In the example shown, eccentric coupler output 728 includes an internal spline 740 that is sized and configured to securely receive a corresponding portion of output puck 601, as described in greater detail below.

Upper ball bearing 726 of the present example includes an inner race 726a, an outer race 726b, and a plurality of bearing balls 726c disposed therebetween. In the example shown, inner race 726a is sized and configured to securely receive eccentric coupler output 728 at a position above spacer 724 such that inner race 726a may rotate together with eccentric coupler output; while outer race 726b may be sized and configured to be fixedly secured to another portion of the tool driver 500, such as to housing 505, such that outer race 726b may remain static relative to housing 505. Thus, upper ball bearing 726 may be substantially centered relative to the central axis CA, and may minimize rolling resistance between eccentric coupler output 728 and the housing of the respective instrument carriage 353.

Lower ball bearing 722 of the present example includes an inner race 722a, an outer race 722b, and a plurality of bearing balls 722c disposed therebetween. In the example shown, inner race 722a is sized and configured to securely receive stem 730 such that inner race 722a may rotate together with stem 730; while outer race 722b is sized and configured to be securely received within central lower sleeve portion 624 of stator housing bottom 610 such that outer race 722b may remain static relative to stator housing bottom 610. Thus, lower ball bearing 722 may be substantially centered relative to the central axis CA, and may minimize rolling resistance between stem 730 and housing assembly 602.

While eccentric coupler assembly 608 of the present example includes lower ball bearing 722 and upper ball bearing 726, it will be appreciated that eccentric coupler assembly 608 may include any other suitable type(s) and/or quantity of bearings. For example, any other suitable type of rolling-element bearing(s) may be used in place of any one or more of lower ball bearing 722 and upper ball bearing 726.

Output puck 601 of the present example includes an external spline 750 that is sized and configured to be securely received within internal spline 740 of eccentric coupler output 728, such that output puck 601 is configured to rotate together with eccentric coupler output 728 (and thus together with eccentric coupler input 720). In the example shown, compression spring 729 is configured to resiliently bias output puck 601 in an upward direction. For example, spring 729 may be received within a central bore 752 of external spline 750, and may extend between an upwardly-facing surface of head 732 of eccentric coupler input 720 and a downwardly-facing surface of output puck 601. In the example shown, output puck 601 may provide actuator 600 with a reduced height, such as via the illustrated nesting of various components of output puck 601.

In an example of a method of use, rotor shaft 672 may initially be in a respective first rotational position in which cycloid disc 606 is at a first eccentric position relative to the central axis (e.g., offset toward a first side of the central axis CA) such that eccentric coupler input 720 (and thus eccentric coupler output 728 and drive output 601) is in a respective first rotational position, as shown in FIGS. 13A and 14A. Rotor 670 may then be selectively rotated by stator 611 to thereby rotate input portion 681 of rotor shaft 672 about the central axis CA while rotating output portion 686 of rotor shaft 672 eccentrically relative to the central axis CA. As a result, output portion 686 of rotor shaft 672 may drive eccentric movement of the eccentrically-mounted upper ball bearing 673 relative to the central axis CA, which may in turn drive eccentric movement of cycloid disc 606 relative to the central axis CA; and the selective engagement between teeth 642 of spline plate 614 and teeth 714 of cycloid disc 606 may guide such eccentric movement of cycloid disc 606 such that cycloid disc 606 may oscillate radially at a first speed while also rotating at a second speed less than the first speed. Such eccentric movement of cycloid disc 606 may be converted into rotation of drive output 601 about the central axis via eccentric coupler assembly 608. More particularly, at least some bushing surfaces 718 of cycloid disc 606 may transmit the rotation of cycloid disc 606 directly to eccentric coupler input 720 via the corresponding bushing surfaces 736 so that eccentric coupler input 720 may also rotate at the second speed, without transmitting any radial motion of cycloid disc 606 to eccentric coupler input 720. Thus, rotor shaft 672 may be rotated to a respective second rotational position in which cycloid disc 606 is at a second eccentric position relative to the central axis (e.g., offset toward a second side of the central axis CA) such that eccentric coupler input 720 (and thus eccentric coupler output 728 and drive output 601) is in a respective second rotational position, as shown in FIGS. 13B and 14B.

In this manner, the eccentrically-mounted upper ball bearing 673, cycloid disc 606, eccentric coupler input 720, and eccentric coupler output 728 cooperate with each other to at least partially define a compact transmission that provides a substantially increased output torque to drive output 601, at least relative to the input torque provided to the eccentrically-mounted upper ball bearing 673 by rotor shaft 672; and that provides a substantially decreased output speed to drive output 601, at least relative to the input speed provided to the eccentrically-mounted upper ball bearing 673 by rotor shaft 672.

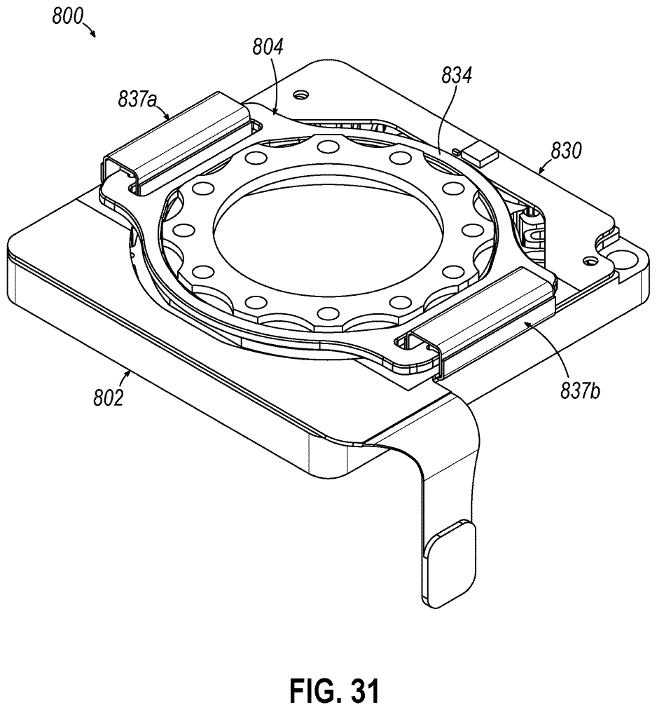

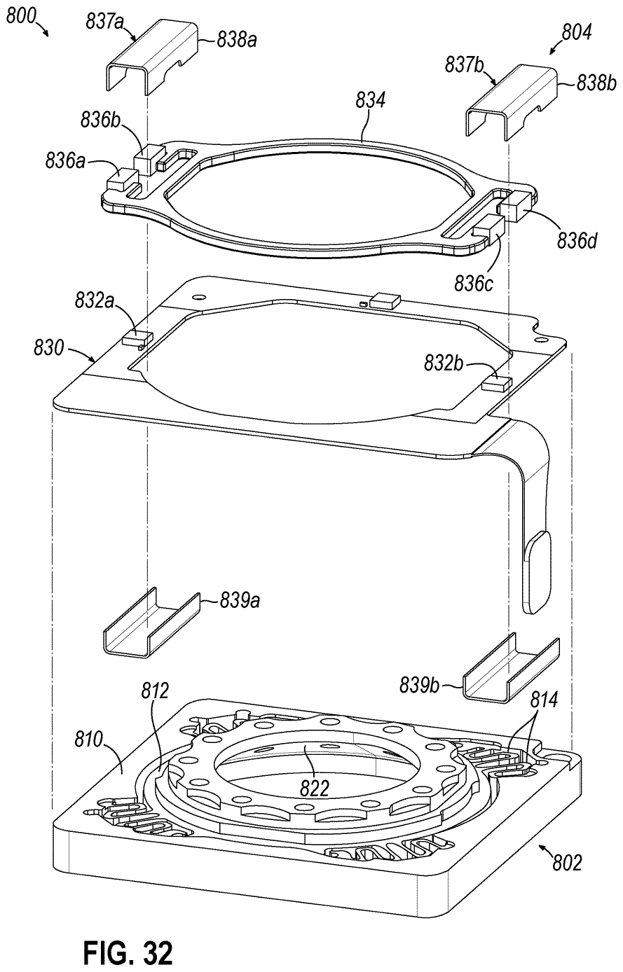

FIGS. 31-32 depict an example of a top cover assembly 800 of an actuator 600 of a tool driver 500, in accordance with some embodiments. Top cover assembly 800 may be incorporated into actuator 600 in place of top cover 616, for example.

As illustrated, top cover assembly 800 includes a top cover 802 and a tunneling magnetoresistance (TMR) torque sensing assembly 804 that is configured to facilitate monitoring of torque within actuator 600. These and other functionalities of top cover assembly 800 are described in greater detail below.

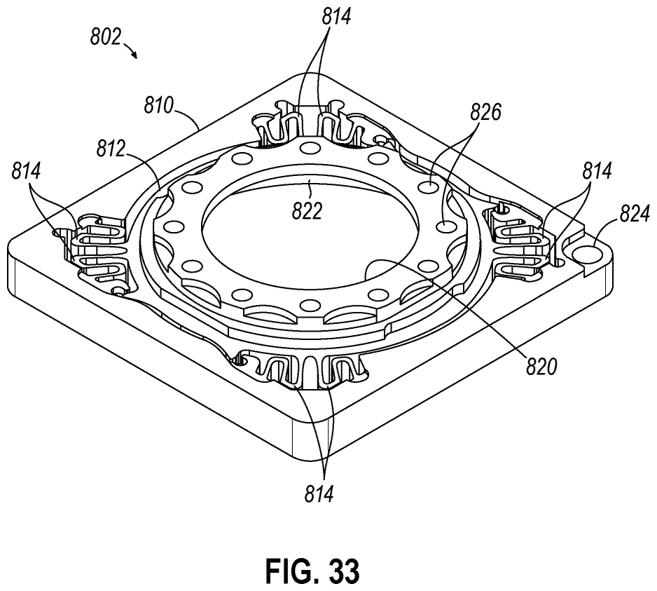

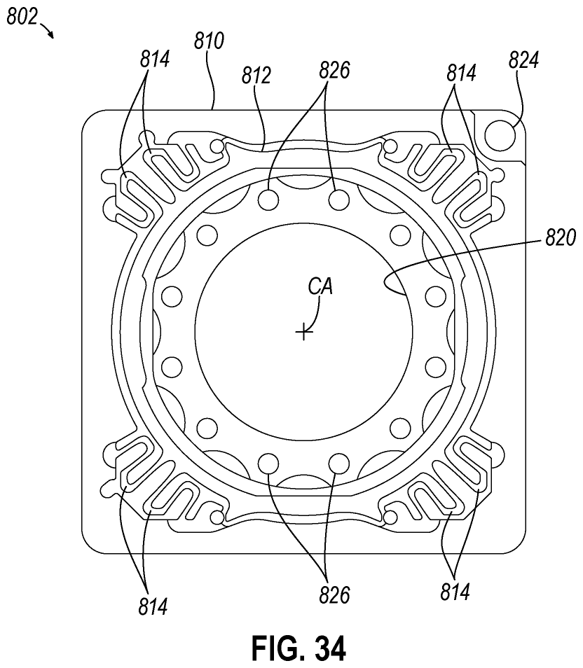

As shown in FIGS. 33-34, top cover 802 of the present example is configured as a flexure, and includes a radially outer, first rigid portion 810 and a radially inner, second rigid portion 812 coupled to each other via a plurality of flexible bridges 814. Flexible bridges 814 may be configured to permit at least some degree of relative rotational motion between first rigid portion 810 and second rigid portion 812 (e.g., about the central axis CA), such as in response to application of a threshold torque to one of first rigid portion 810 or second rigid portion 812.

Second rigid portion 812 of the present example has a generally circular profile (e.g., when viewed from above), and includes a generally circular central bore 820 that is configured to be substantially centered relative to the central axis CA and that is sized and configured to permit at least a portion of eccentric coupler assembly 608 to pass therethrough. Second rigid portion 812 of the present example also includes a generally frustoconical recess 822 that is provided in a lower surface of second rigid portion 812 radially outwardly relative to central bore 820 and that is sized and configured to accommodate at least a portion of eccentric coupler assembly 608.

First rigid portion 810 of the example shown has a generally square-shaped profile (e.g., when viewed from above), and includes at least one bore 824 positioned near an outer periphery (e.g., near a respective corner) of first rigid portion 810 and configured to receive at least one corresponding fastener 619 for fixedly securing first rigid portion 810 to stator housing bottom 610, stator housing top 612, spline plate 614, and guard shim 615.

In the example shown, second rigid portion 812 also includes a generally circular array of threaded bores 826 positioned radially outwardly relative to central bore 820 and configured to threadably engage additional fasteners (not shown) for fixedly securing second rigid portion 812 to another portion of the tool driver 500, such as to housing 505.

Thus, first rigid portion 810 may be fixedly secured to stator housing bottom 610, stator housing top 612, spline plate 614, and guard shim 615, while second rigid portion may be fixedly secured to housing 505; and flexible bridges 814 may permit at least some degree of relative rotational motion between first rigid portion 810 (e.g., together with stator housing bottom 610, stator housing top 612, spline plate 614, and guard shim 615) and second rigid portion 812 (e.g., together with housing 505). As described in greater detail below, torque sensing assembly 804 may be configured to facilitate monitoring of torque within actuator 600 based on such relative rotational motion between first rigid portion 810 and second rigid portion 812.

Torque sensing assembly 804 of the present example includes a printed circuit board (“PCB”) 830 having a plurality of (e.g., two) magnetic sensors in the form of first and second low-hysteresis magnetic sensors 832a, 832b; a mounting ring 834 carrying a plurality of (e.g., four) magnets 836a, 836b, 836c, 836d arranged in opposing pairs; and a plurality of (e.g., two) magnetic shielding tunnels 837a, 837b, each including a respective upper portion 838a, 838b and a respective lower portion 839a, 839b.

PCB 830 (including magnetic sensors 832) of the present example is fixedly secured to first rigid portion 810 of top cover 802, while mounting ring 834 (carrying magnets 836) of the present example is fixedly secured to second rigid portion 812 of top cover 802. In the example shown, magnetic sensors 832a, 832b are disposed opposite from each other (e.g., relative to the central axis CA). A first pair of magnets 836a, 836b is disposed opposite from a second pair of magnets 836c, 836d (e.g., relative to the central axis CA), with first magnetic sensor 832a being interposed between the first pair of magnets 836a, 836b, and with second magnetic sensor 832b being interposed between the second pair of magnets 836c, 836d. Each respective pair of magnets 836a, 836b, 836c, 836d may be arranged with like poles facing each other to thereby generate a magnetic field with a linear region about a centrally located zero field position between the respective pair of magnets 836a, 836b, 836c, 836d. Each magnetic sensor 832a, 832b may be configured to locate at the corresponding zero field position between the respective pair of magnets 836a, 836b, 836c, 836d in the absence of application of a threshold torque between first and second rigid portions 810, 812; and to be displaced from the corresponding zero field position between the respective pair of magnets 836a, 836b, 836c, 836d in response to application of the threshold torque between first and second rigid portions 810, 812. Magnetic sensors 832a, 832b may be configured to generate one or more signals indicative of their respective displacements, and to send such signals to control system 145. For example, PCB 830 may be communicatively coupled to control system 145, such as via PCA 618. Processing circuitry of the control system 145 may be configured to determine the displacements of magnetic sensors 832a, 832b based on the signals received from magnetic sensors 832a, 832b; and to correlate such displacements to the torque being applied between first and second rigid portions 810, 812. Thus, control system 145 may accurately determine the torque being applied between first rigid portion 810 (e.g., together with stator housing bottom 610, stator housing top 612, spline plate 614, and guard shim 615) and second rigid portion 812 (e.g., together with housing 505), based on the signals received from magnetic sensors 832a, 832b.

While top cover assembly 800 of the present example includes TMR torque sensing assembly 804, top cover assembly 800 may alternatively include any other suitable type of torque sensing assembly for monitoring torque applied between first and second rigid portions 810, 812 of top cover 802. For example, top cover assembly 800 may include one or more Hall effect sensors (not shown) for monitoring torque applied between first and second rigid portions 810, 812 of top cover 802.

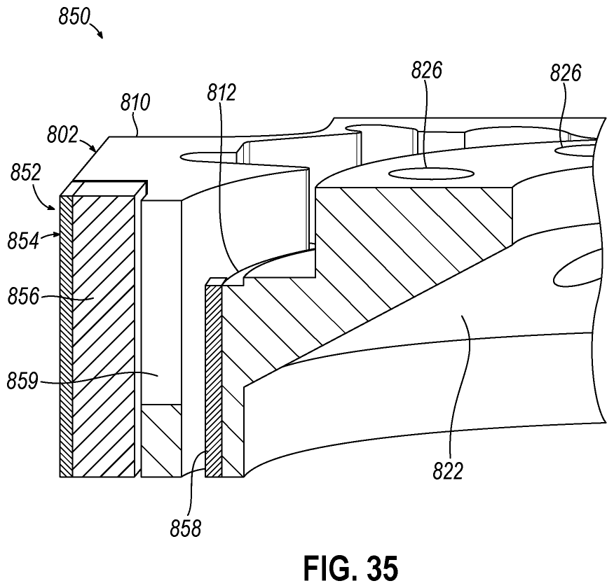

FIG. 35 depicts an example of a top cover assembly 850 of an actuator 600 of a tool driver 500, in accordance with some embodiments. Top cover assembly 850 may be incorporated into actuator 600 in place of top cover 616, for example.

As illustrated, top cover assembly 850 includes top cover 802 and an optical torque sensing assembly 852 that is configured to facilitate monitoring of torque within actuator 600. These and other functionalities of top cover assembly 850 are described in greater detail below.

Torque sensing assembly 852 of the present example includes a printed circuit board (“PCB”) 854 having an optical sensor in the form of an encoder integrated circuit (IC) 856; a visible marker in the form of a glass code scale 858; and a window 859 extending radially through a wall of top cover 802 between encoder IC 856 and scale 858.

PCB 854 (including encoder IC 856) of the present example is fixedly secured to first rigid portion 810 of top cover 802, while scale 858 of the present example is fixedly secured to second rigid portion 812 of top cover 802. In the example shown, encoder IC 856 confronts scale 858 (e.g., through window 859) in a radial direction (e.g., perpendicular to central axis CA). Scale 858 may be configured to radially align with encoder IC 856 in the absence of application of a threshold torque between first and second rigid portions 810, 812; and to be radially misaligned (e.g., displaced) from encoder IC 856 in response to application of the threshold torque between first and second rigid portions 810, 812. Encoder IC 856 may be configured to generate one or more signals indicative of such displacement, and to send such signals to control system 145. For example, PCB 854 may be communicatively coupled to control system 145, such as via PCA 618. Processing circuitry of the control system 145 may be configured to determine the displacement of scale 858 based on the signals received from encoder IC 856; and to correlate such displacement to the torque being applied between first and second rigid portions 810, 812. Thus, control system 145 may accurately determine the torque being applied between first rigid portion 810 (e.g., together with stator housing bottom 610, stator housing top 612, spline plate 614, and guard shim 615) and second rigid portion 812 (e.g., together with housing 505), based on the signals received from encoder IC 856.

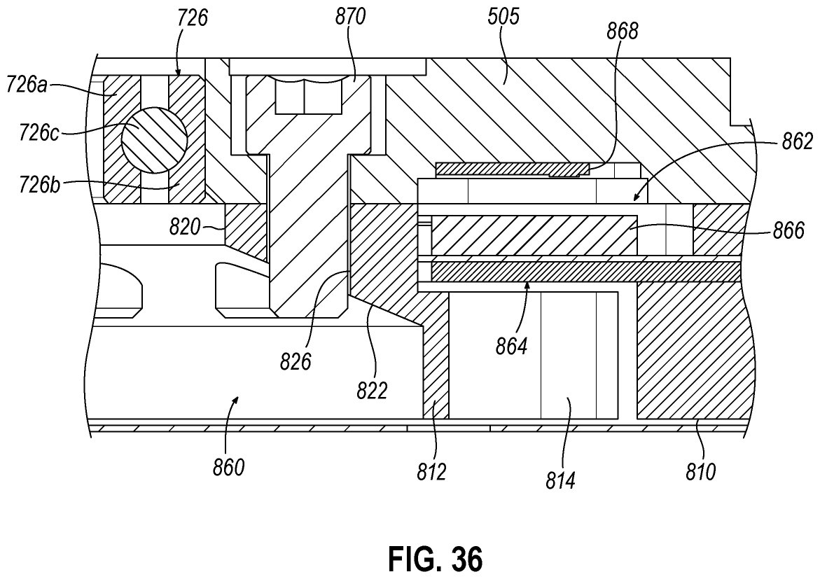

FIG. 36 depicts an example of a top cover assembly 860 of an actuator 600 of a tool driver 500, in accordance with some embodiments. Top cover assembly 860 may be incorporated into actuator 600 in place of top cover 616, for example.

As illustrated, top cover assembly 860 includes top cover 802 and an optical torque sensing assembly 862 that is configured to facilitate monitoring of torque within actuator 600. These and other functionalities of top cover assembly 860 are described in greater detail below.

Torque sensing assembly 862 of the present example includes a printed circuit board (“PCB”) 864 having an optical sensor in the form of an encoder integrated circuit (IC) 866; and a visible marker in the form of a glass code scale 868.

PCB 864 (including encoder IC 866) of the present example is fixedly secured to first rigid portion 810 of top cover 802, while scale 868 of the present example is fixedly secured to second rigid portion 812 of top cover 802. More particularly, scale 868 of the present example is fixedly secured to housing 505 (e.g., to a mounting plate of housing 505), which is in turn fixedly secured to second rigid portion 812 via one or more additional fasteners in the form of bolts 870 that are threadably engaged with corresponding threaded bores 826. In the example shown, encoder IC 866 confronts scale 868 in an axial direction (e.g., parallel to central axis CA). Scale 868 may be configured to axially align with encoder IC 866 in the absence of application of a threshold torque between first and second rigid portions 810, 812; and to be axially misaligned (e.g., displaced) from encoder IC 866 in response to application of the threshold torque between first and second rigid portions 810, 812. Encoder IC 866 may be configured to generate one or more signals indicative of such displacement, and to send such signals to control system 145. For example, PCB 864 may be communicatively coupled to control system 145, such as via PCA 618. Processing circuitry of the control system 145 may be configured to determine the displacement of scale 868 based on the signals received from encoder IC 866; and to correlate such displacement to the torque being applied between first and second rigid portions 810, 812. Thus, control system 145 may accurately determine the torque being applied between first rigid portion 810 (e.g., together with stator housing bottom 610, stator housing top 612, spline plate 614, and guard shim 615) and second rigid portion 812 (e.g., together with housing 505), based on the signals received from encoder IC 866.

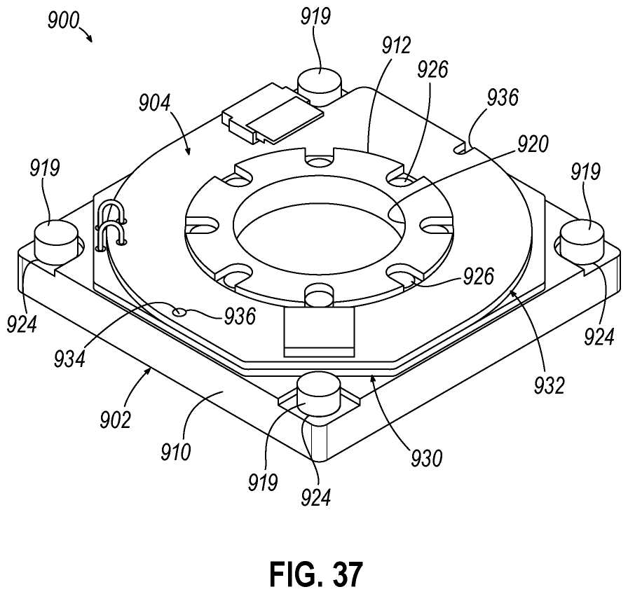

FIG. 37 depicts an example of a top cover assembly 900 of an actuator 600 of a tool driver 500, in accordance with some embodiments. Top cover assembly 900 may be incorporated into actuator 600 in place of top cover 616, for example.

As illustrated, top cover assembly 900 includes a top cover 902 and a capacitive torque sensing assembly 904 that is configured to facilitate monitoring of torque within actuator 600. These and other functionalities of top cover assembly 900 are described in greater detail below.

Top cover 902 of the present example may be similar to top cover 802 described above, except as otherwise described below. In this regard, top cover 902 is configured as a flexure, and includes a radially outer, first rigid portion 910 and a radially inner, second rigid portion 912 which may be coupled to each other via a plurality of flexible bridges (not shown) similar to flexible bridges 814, to permit at least some degree of relative rotational motion between first rigid portion 910 and second rigid portion 912 (e.g., about the central axis CA), such as in response to application of a threshold torque to one of first rigid portion 910 or second rigid portion 912.

Second rigid portion 912 of the present example has a generally circular profile (e.g., when viewed from above), and includes a generally circular central bore 920 that is configured to be substantially centered relative to the central axis CA and that is sized and configured to permit at least a portion of eccentric coupler assembly 608 to pass therethrough.

First rigid portion 910 of the example shown has a generally square-shaped profile (e.g., when viewed from above), and includes a plurality of (e.g., four) bores 924 positioned near an outer periphery (e.g., near respective corners) of first rigid portion 910 and configured to receive at least one corresponding fastener 919 for fixedly securing first rigid portion 910 to stator housing bottom 610, stator housing top 612, spline plate 614, and guard shim 615.

In the example shown, second rigid portion 912 also includes a generally circular array of threaded bores 926 positioned radially outwardly relative to central bore 920 and configured to threadably engage additional fasteners (not shown) for fixedly securing second rigid portion 912 to another portion of the tool driver 500, such as to housing 505.

Torque sensing assembly 904 of the present example includes an input printed circuit board (“PCB”) 930 adhered to top cover 902; an upper printed circuit board (“PCB”) 932 positioned above input PCB 930; a pair of pins 934 (one shown) disposed opposite from each other (e.g., relative to the central axis CA), and received in corresponding openings 936 in PCB's 930, 932 and slip fit into top cover 902. Torque sensing assembly 904 may also include any suitable number of shims (not shown) for setting an appropriate air gap.

Capacitive torque sensing assembly 904 may be configured to generate one or more signals indicative of any displacement of one or more ground pads on either PCB 930, 932, and to send such signals to control system 145. For example, PCB's 930, 932 may be communicatively coupled to control system 145, such as via PCA 618. Processing circuitry of the control system 145 may be configured to determine the displacement of the one or more ground pads based on signals received from PCB's 930, 932; and to correlate such displacement to the torque being applied between first and second rigid portions 910, 912. Thus, control system 145 may accurately determine the torque being applied between first rigid portion 910 (e.g., together with stator housing bottom 610, stator housing top 612, spline plate 614, and guard shim 615) and second rigid portion 912 (e.g., together with housing 505), based on the signals received from capacitive torque sensing assembly 904.

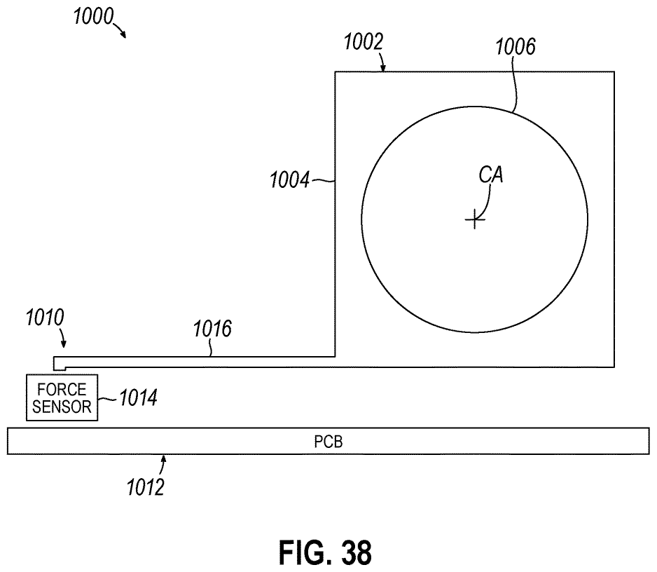

FIG. 38 depicts another example of a portion of a tool driver 1000 that may be incorporated into distal assembly 330 in place of tool driver 332, in accordance with some embodiments. Tool driver 1000 may be similar to tool driver 500 described above, except as otherwise described below.

As illustrated, tool driver 1000 includes at least one actuator 1002, which may be similar to actuator 600 described above. Actuator 1002 may be housed within a housing (not shown) of tool driver 1000, such as housing 505. In the example shown, actuator 1002 includes a housing assembly 1004 and a drive output 1006, which may be similar to housing assembly 602 and drive output 601 described above, respectively. Actuator 1002 may also include a rotor assembly (not shown), a cycloid disc (not shown), and an eccentric coupler assembly (not shown), similar to rotor assembly 604, and cycloid disc 606, eccentric coupler assembly 608 described above, respectively. In the example shown, tool driver 1000 also includes a torque sensing assembly 1010.

Torque sensing assembly 1010 of the present example includes a printed circuit board (“PCB”) 1012 having at least one force sensor 1014; and at least one lever arm 1016 configured to apply a force to the at least one force sensor 1014.

PCB 1012 of the present example may be fixedly secured to an interior of housing 505, while lever arm 1016 extends laterally outwardly from housing assembly 1004. Force sensor 1014 may be configured to generate one or more signals indicative of the force applied to force sensor 1014 by lever arm 1016, and to send such signals to control system 145. For example, PCB 1012 may be communicatively coupled to control system 145, such as via PCA 618. Processing circuitry of the control system 145 may be configured to determine the force applied to force sensor 1014 based on the signals received from force sensor 1014; and to correlate such displacements to the torque being applied between housing assembly 1004 of actuator 1002 and housing 505 of tool driver 1000. Thus, control system 145 may accurately determine the torque being applied between housing assembly 1004 of actuator 1002 and housing 505 of tool driver 1000 based on the signals received from force sensor 1014.

In some versions, torque sensing assembly 1010 may be configured to sense torque in two directions. For example, force sensor 1014 may be resiliently biased with a spring such that a torque in the opposite direction can be measured as a reduction in preload. As another example, an additional force sensor may be positioned opposite from force sensor 1014 (e.g., relative to the central axis CA) and an additional lever arm may be configured to apply a force to the additional force sensor.

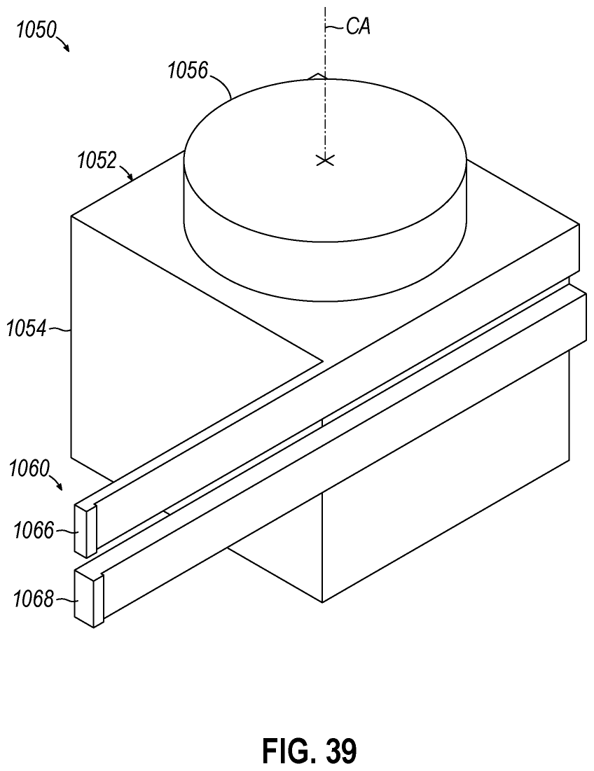

FIG. 39 depicts another example of a portion of a tool driver 1050 that may be incorporated into distal assembly 330 in place of tool driver 332, in accordance with some embodiments. Tool driver 1050 may be similar to tool driver 1000 described above, except as otherwise described below.

As illustrated, tool driver 1050 includes at least one actuator 1052, which may be similar to actuator 600 described above. Actuator 1052 may be housed within a housing (not shown) of tool driver 1050, such as housing 505. In the example shown, actuator 1052 includes a housing assembly 1054 and a drive output 1056, which may be similar to housing assembly 602 and drive output 601 described above, respectively. Actuator 1052 may also include a rotor assembly (not shown), a cycloid disc (not shown), and an eccentric coupler assembly (not shown), similar to rotor assembly 604, and cycloid disc 606, eccentric coupler assembly 608 described above, respectively. In the example shown, tool driver 1050 also includes a torque sensing assembly 1060.

Torque sensing assembly 1060 of the present example includes a printed circuit board (not shown) similar to PCB 1012 having at least one force sensor similar to force sensor 1014; and at least one lever arm 1066 configured to apply a force to the at least one force sensor 1014. In the example shown, an auxiliary lever arm 1068 extends parallel to lever arm 1066 and may be fixedly secured to housing 505 to carry a portion of the load, thereby reducing the amount of force applied to force sensor 1014 by lever arm 1066.

Various principles of this technology are described with reference to laparoscopic procedures, where surgical instruments are introduced to a patient's abdomen through laparoscopic incisions, and endoscopic procedures, where surgical instruments are introduced through natural orifices. In some variations, a configuration of a surgical system and/or a method of use can involve various types of procedures, anatomical locations, and/or anatomical openings for introducing instruments into a body. Various aspects of the subject matter described herein may be applied to, for instance, laparoscopic procedures, endoscopic procedures, endoluminal procedures, thoracic procedures, and/or procedures involving combinations of any two or more of these approaches.

Various principles of this technology are described with reference to rigid and flexible instrumentation, where, for instance, rigid instruments are introduced through incisions or laparoscopic ports, and flexible instruments are introduced through natural orifices. In various configurations, any one or more of these instruments may be flexible or rigid, such as, for example one or more rigid instruments or rigid scopes introduced through a natural orifice and/or one/or more flexible instruments or flexible scopes introduced through an incision or a laparoscopic port.

Various examples disclosed herein describe usage of a surgical system to perform a procedure on a patient, wherein instruments are inserted into a body of the patient. In various configurations, the system may be used, for instance, in educational or lab settings, where a body portion of a model, cadaver, animal, or inanimate object is placed upon the headrest. Such methods may be useful for surgeon training, product testing, development applications, or the like. Accordingly, it will be understood that methods described herein are not limited to medical procedures performed on a human body but can be implemented on bodies or objects that are not part of a live patient or human.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for illustrative purposes. It is contemplated that the various teachings herein may be arranged and applied in numerous other ways. It is also contemplated that some variations may omit certain features referred to in the examples below. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A tool driver for a robotic surgical system, the tool driver comprising: a housing, a drive output movably mounted to the housing and configured to operatively engage a drive input of a surgical instrument, and an actuator including: an internal ring gear including a first plurality of involute teeth, the internal ring gear being centered relative to a central axis, a cycloid disc positioned eccentrically relative to the central axis, the cycloid disc including:
a second plurality of involute teeth configured to selectively engage the first plurality of involute teeth, and a first plurality of bushing surfaces, a rotor configured to drive eccentric movement of the cycloid disc relative to the central axis, and an eccentric coupler including:
   an eccentric coupler input having a second plurality of bushing surfaces configured to selectively engage the first plurality of bushing surfaces to thereby convert eccentric movement of the cycloid disc relative to the central axis into rotation of the eccentric coupler about the central axis, and an eccentric coupler output configured to operatively engage the drive output to thereby transmit rotation of the eccentric coupler about the central axis to the drive output.

### Example 2

The tool driver of Example 1, further comprising a rotor shaft fixedly secured to the rotor, the rotor shaft including a rotor output portion, the rotor output portion being eccentric relative to the central axis.

### Example 3

The tool driver of Example 2, further comprising a bearing mounted to the rotor output portion and configured to transmit eccentric movement of the rotor output portion to the cycloid disc.

### Example 4

The tool driver of Example 3, further comprising a pair of balancing discs mounted to the rotor output portion, the bearing being sandwiched between the pair of balancing discs.

### Example 5

The tool driver of any of Examples 2 through 3, further comprising a bearing mounted within a central bore of the rotor shaft, at least a portion of the eccentric coupler extending through the bearing.

### Example 6

The tool driver of any of Examples 1 through 5, the cycloid disc including a plurality of receptacles, each receptacle of the plurality of receptacles defining a respective bushing surface of the first plurality of bushing surfaces.

### Example 7

The tool driver of Example 6, the eccentric coupler input including a plurality of pins, each pin of the plurality of pins being configured to be received within a corresponding receptacle of the plurality of receptacles, each pin of the plurality of pins defining a respective bushing surface of the second plurality of bushing surfaces.

### Example 8

The tool driver of Example 7, each pin of the plurality of pins being sized to move at least one of radially or circumferentially relative to the corresponding receptacle of the plurality of receptacles.

### Example 9

The tool driver of any of Examples 7 through 8, the eccentric coupler input including a stem and a head extending radially outwardly from an upper end of the stem, the plurality of pins extending downwardly from an outer periphery of the head.

### Example 10

The tool driver of any of Examples 1 through 9, further comprising: a flexure including:
a first rigid portion fixedly secured against movement relative to the internal ring gear of the actuator, a second rigid portion fixedly secured against movement relative to the housing of the tool driver, and at least one flexible bridge extending between the first rigid portion and the second rigid portion; and a torque sensing assembly configured to detect relative rotational movement between the first rigid portion and the second rigid portion about the central axis.

### Example 11

The tool driver of Example 10, the torque sensing assembly including a magnetic torque sensing assembly.

### Example 12

The tool driver of Example 11, the magnetic torque sensing assembly including a tunneling magnetoresistance torque sensing assembly.

### Example 13

The tool driver of Example 12, the tunneling magnetoresistance torque sensing assembly including: at least one magnetic sensor fixedly secured to one of the first or second rigid portions, at least one pair of magnets fixedly secured to the other of the first or second rigid portions, the at least one magnetic sensor being interposed between the at least one pair of magnets, and at least one magnetic shielding tunnel surrounding the at least one pair of magnets.

### Example 14

The tool driver of Example 13, the at least one magnetic sensor including first and second magnetic sensors positioned opposite from each other relative to the central axis, the at least one pair of magnets including first and second pairs of magnets, the first magnetic sensor being interposed between the first pair of magnets, the second magnetic sensor being interposed between the second pair of magnets, the at least one magnetic shielding tunnel including first and second magnetic shielding tunnels, the first magnetic shielding tunnel surrounding the first pair of magnets, the second magnetic shielding tunnel surrounding the second pair of magnets.

### Example 15

A robotic surgical system comprising: the tool driver of any of Examples 1 through 14; and a surgical instrument including: an instrument base, an elongate shaft extending distally from the instrument base, and a drive input movably mounted to the instrument base, the drive output of the tool driver being configured to operatively engage the drive input of the surgical instrument.

### Example 16

A tool driver for a robotic surgical system, the tool driver comprising: a housing; a drive output movably mounted to the housing and configured to operatively engage a drive input of a surgical instrument; and an actuator including: a rotor operatively coupled to the drive output, a stator configured to selectively rotate the rotor about a central axis; a flexure including: a first rigid portion fixedly secured against movement relative to the stator of the actuator, a second rigid portion fixedly secured against movement relative to the housing of the tool driver, and at least one flexible bridge extending between the first rigid portion and the second rigid portion; and a torque sensing assembly configured to detect relative rotational movement between the first rigid portion and the second rigid portion about the central axis.

### Example 17

The tool driver of Example 16, the torque sensing assembly including a magnetic torque sensing assembly.

### Example 18

The tool driver of Example 17, the magnetic torque sensing assembly including a tunneling magnetoresistance torque sensing assembly.

### Example 19

A robotic surgical system comprising: the tool driver of any of Examples 16 through 18; and a surgical instrument including: an instrument base, an elongate shaft extending distally from the instrument base, and a drive input movably mounted to the instrument base, the drive output of the tool driver being configured to operatively engage the drive input of the surgical instrument.

### Example 20

A tool driver for a robotic surgical system, the tool driver comprising: a housing; a drive output movably mounted to the housing and configured to operatively engage a drive input of a surgical instrument; an actuator including: a housing assembly including: an internal ring gear centered relative to a central axis, and a top cover including: a first rigid portion fixedly secured against movement relative to the internal ring gear, a second rigid portion fixedly secured against movement relative to the housing of the tool driver, and at least one flexible bridge extending between the first rigid portion and the second rigid portion, a cycloid disc positioned eccentrically relative to the central axis, the cycloid disc including an external ring gear configured to selectively engage the internal ring gear, a rotor configured to drive eccentric movement of the cycloid disc relative to the central axis, an eccentric coupler configured to convert eccentric movement of the cycloid disc relative to the central axis into rotation of the drive output about the central axis; and a torque sensing assembly configured to detect relative rotational movement between the first rigid portion and the second rigid portion about the central axis.

### Example 21

An actuator for a surgical tool driver, the actuator comprising: an internal ring gear including a first plurality of involute teeth, the internal ring gear being centered relative to a central axis; a cycloid disc positioned eccentrically relative to the central axis, the cycloid disc including: a second plurality of involute teeth configured to selectively engage the first plurality of involute teeth, and a first plurality of bushing surfaces; a rotor configured to drive eccentric movement of the cycloid disc relative to the central axis; and an eccentric coupler including: an eccentric coupler input having a second plurality of bushing surfaces configured to selectively engage the first plurality of bushing surfaces to thereby convert eccentric movement of the cycloid disc relative to the central axis into rotation of the eccentric coupler about the central axis, and an eccentric coupler output configured to operatively engage a drive output to thereby transmit rotation of the eccentric coupler about the central axis to the drive output.

### Example 22

The actuator of Example 21, further comprising a rotor shaft fixedly secured to the rotor, the rotor shaft including a rotor output portion, the rotor output portion being eccentric relative to the central axis.

### Example 23

The actuator of Example 22, further comprising a bearing mounted to the rotor output portion and configured to transmit eccentric movement of the rotor output portion to the cycloid disc.

### Example 24

The actuator of Example 23, further comprising a pair of balancing discs mounted to the rotor output portion, the bearing being sandwiched between the pair of balancing discs.

### Example 25

The actuator of any of Examples 22 through 24, further comprising a bearing mounted within a central bore of the rotor shaft, at least a portion of the eccentric coupler extending through the bearing.

### Example 26

The actuator of any of Examples 21 through 25, the cycloid disc including a plurality of receptacles, each receptacle of the plurality of receptacles defining a respective bushing surface of the first plurality of bushing surfaces.

### Example 27

The actuator of Example 26, the eccentric coupler input including a plurality of pins, each pin of the plurality of pins being configured to be received within a corresponding receptacle of the plurality of receptacles, each pin of the plurality of pins defining a respective bushing surface of the second plurality of bushing surfaces.

### Example 28

The actuator of Example 27, each pin of the plurality of pins being sized to move at least one of radially or circumferentially relative to the corresponding receptacle of the plurality of receptacles.

### Example 29

The actuator of any of Examples 27 through 28, the eccentric coupler input including a stem and a head extending radially outwardly from an upper end of the stem, the plurality of pins extending downwardly from an outer periphery of the head.

### Example 30

The actuator of any of Examples 21 through 29, further comprising: a flexure including: a first rigid portion fixedly secured against movement relative to the internal ring gear of the actuator, a second rigid portion configured to be fixedly secured against movement relative to a housing of the surgical tool driver, and at least one flexible bridge extending between the first rigid portion and the second rigid portion; and a torque sensing assembly configured to detect relative rotational movement between the first rigid portion and the second rigid portion about the central axis.

### Example 31

An actuator for a surgical tool driver, the actuator comprising: a rotor configured to be operatively coupled to a drive output of the surgical tool driver; a stator configured to selectively rotate the rotor about a central axis; a flexure including: a first rigid portion fixedly secured against movement relative to the stator of the actuator, a second rigid portion configured to be fixedly secured against movement relative to a housing of the surgical tool driver, and at least one flexible bridge extending between the first rigid portion and the second rigid portion; and a torque sensing assembly configured to detect relative rotational movement between the first rigid portion and the second rigid portion about the central axis.

### Example 32

The actuator of Example 31, the torque sensing assembly including a magnetic torque sensing assembly.

### Example 33

The actuator of Example 32, the magnetic torque sensing assembly including a tunneling magnetoresistance torque sensing assembly.

### Example 34

The actuator of Example 33, the tunneling magnetoresistance torque sensing assembly including: at least one magnetic sensor fixedly secured to one of the first or second rigid portions, at least one pair of magnets fixedly secured to the other of the first or second rigid portions, the at least one magnetic sensor being interposed between the at least one pair of magnets, and at least one magnetic shielding tunnel surrounding the at least one pair of magnets.

### Example 35

The actuator of Example 34, the at least one magnetic sensor including first and second magnetic sensors positioned opposite from each other relative to the central axis, the at least one pair of magnets including first and second pairs of magnets, the first magnetic sensor being interposed between the first pair of magnets, the second magnetic sensor being interposed between the second pair of magnets, the at least one magnetic shielding tunnel including first and second magnetic shielding tunnels, the first magnetic shielding tunnel surrounding the first pair of magnets, the second magnetic shielding tunnel surrounding the second pair of magnets.

### Example 36

The actuator of any of Examples 34 through 35, the at least one magnetic sensor being disposed on a printed circuit board.

### Example 37

The actuator of any of Examples 34 through 36, the at least one pair of magnets being carried by a mounting ring.

### Example 38

The actuator of any of Examples 34 through 37, the at least one magnetic sensor being fixedly secured to the first rigid portion, the at least one pair of magnets being fixedly secured to the second rigid portion.

### Example 39

The actuator of any of Examples 31 through 38, the first rigid portion being positioned radially outwardly from the second rigid portion.

### Example 40

The actuator of any of Examples 31 through 39, the flexure defining a top cover of a housing assembly of the actuator.

Use of “or” is intended in the inclusive rather than exclusive sense, unless explicitly stated otherwise or the context clearly dictates otherwise. Thus, for example, reference to “A” or “B” can encompass “A” only, “B” only, or both “A” and “B.” As another example, reference to “A, B, or C” can encompass “A” only, “B” only, “C” only, or any combination of two or more of “A” or “B” or “C.” Accordingly, the term “or” should be generally understood as equivalent to “and/or” unless stated otherwise or the context clearly dictates to the contrary.

It should be appreciated that any specific order of steps shown or described herein is illustrative in nature and should not be construed as required unless explicitly stated or the context clearly dictates otherwise. Thus, for example, with respect to any processes or methods herein, any two or more steps or stages in a method or process may performed serially or in parallel, in any combination, and may be performed in any order, unless explicitly stated or the context clearly dictates otherwise.

In some instances, relative positions or orientations are used, such as top, bottom, upper, lower, forward, backward, front, rear, left, right, up down, horizontal, vertical, longitudinal, lateral, or the like. These terms may be used to refer to an arbitrary frame of reference or a frame of reference shown in the drawings, for purposes of explanation or to demonstrate the relative spatial configurations associated with various elements. These terms should not be understood to require any particular gravitational or other frame of reference unless explicitly stated or the context clearly dictates otherwise.

To the extent any headings are used through this description, these headings are used for convenience only and should not be construed as limit the scope of disclosure or the description under a heading to only the topic associated with the heading in anyway.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various examples, configurations, or embodiments of the present technology, further adaptations of the systems or methods described herein may be accomplished by appropriate modifications by one of ordinary skill in the art without departing from the scope of the technology described herein. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the claimed subject matter should be considered in terms of the following claims and is understood not to be limited to the details of structure and operation shown and described in the specification and drawings.

1. A tool driver for a robotic surgical system, the tool driver comprising:
a housing,
a drive output movably mounted to the housing and configured to operatively engage a drive input of a surgical instrument, and
an actuator including:
an internal ring gear including a first plurality of involute teeth, the internal ring gear being centered relative to a central axis,
a cycloid disc positioned eccentrically relative to the central axis, the cycloid disc including:
a second plurality of involute teeth configured to selectively engage the first plurality of involute teeth, and
a first plurality of bushing surfaces,
a rotor configured to drive eccentric movement of the cycloid disc relative to the central axis, and
an eccentric coupler including:
an eccentric coupler input having a second plurality of bushing surfaces configured to selectively engage the first plurality of bushing surfaces to thereby convert eccentric movement of the cycloid disc relative to the central axis into rotation of the eccentric coupler about the central axis, and
an eccentric coupler output configured to operatively engage the drive output to thereby transmit rotation of the eccentric coupler about the central axis to the drive output.

2. The tool driver of claim 1, further comprising a rotor shaft fixedly secured to the rotor, the rotor shaft including a rotor output portion, the rotor output portion being eccentric relative to the central axis.

3. The tool driver of claim 2, further comprising a bearing mounted to the rotor output portion and configured to transmit eccentric movement of the rotor output portion to the cycloid disc.

4. The tool driver of claim 3, further comprising a pair of balancing discs mounted to the rotor output portion, the bearing being sandwiched between the pair of balancing discs.

5. The tool driver of claim 2, further comprising a bearing mounted within a central bore of the rotor shaft, at least a portion of the eccentric coupler extending through the bearing.

6. The tool driver of claim 1, the cycloid disc including a plurality of receptacles, each receptacle of the plurality of receptacles defining a respective bushing surface of the first plurality of bushing surfaces.

7. The tool driver of claim 6, the eccentric coupler input including a plurality of pins, each pin of the plurality of pins being configured to be received within a corresponding receptacle of the plurality of receptacles, each pin of the plurality of pins defining a respective bushing surface of the second plurality of bushing surfaces.

8. The tool driver of claim 7, each pin of the plurality of pins being sized to move at least one of radially or circumferentially relative to the corresponding receptacle of the plurality of receptacles.

9. The tool driver of claim 7, the eccentric coupler input including a stem and a head extending radially outwardly from an upper end of the stem, the plurality of pins extending downwardly from an outer periphery of the head.

10. The tool driver of claim 1, further comprising:
a flexure including:
a first rigid portion fixedly secured against movement relative to the internal ring gear of the actuator,
a second rigid portion fixedly secured against movement relative to the housing of the tool driver, and
at least one flexible bridge extending between the first rigid portion and the second rigid portion; and
a torque sensing assembly configured to detect relative rotational movement between the first rigid portion and the second rigid portion about the central axis.

11. The tool driver of claim 10, the torque sensing assembly including a magnetic torque sensing assembly.

12. The tool driver of claim 11, the magnetic torque sensing assembly including a tunneling magnetoresistance torque sensing assembly.

13. The tool driver of claim 12, the tunneling magnetoresistance torque sensing assembly including:
at least one magnetic sensor fixedly secured to one of the first or second rigid portions,
at least one pair of magnets fixedly secured to the other of the first or second rigid portions, the at least one magnetic sensor being interposed between the at least one pair of magnets, and
at least one magnetic shielding tunnel surrounding the at least one pair of magnets.

14. The tool driver of claim 13, the at least one magnetic sensor including first and second magnetic sensors positioned opposite from each other relative to the central axis, the at least one pair of magnets including first and second pairs of magnets, the first magnetic sensor being interposed between the first pair of magnets, the second magnetic sensor being interposed between the second pair of magnets, the at least one magnetic shielding tunnel including first and second magnetic shielding tunnels, the first magnetic shielding tunnel surrounding the first pair of magnets, the second magnetic shielding tunnel surrounding the second pair of magnets.

15. A robotic surgical system comprising:
the tool driver of claim 1; and
a surgical instrument including:
an instrument base,
an elongate shaft extending distally from the instrument base, and
a drive input movably mounted to the instrument base,
the drive output of the tool driver being configured to operatively engage the drive input of the surgical instrument.

16. A tool driver for a robotic surgical system, the tool driver comprising:
a housing;
a drive output movably mounted to the housing and configured to operatively engage a drive input of a surgical instrument; and
an actuator including:
a rotor operatively coupled to the drive output,
a stator configured to selectively rotate the rotor about a central axis;
a flexure including:
a first rigid portion fixedly secured against movement relative to the stator of the actuator,
a second rigid portion fixedly secured against movement relative to the housing of the tool driver, and
at least one flexible bridge extending between the first rigid portion and the second rigid portion; and
a torque sensing assembly configured to detect relative rotational movement between the first rigid portion and the second rigid portion about the central axis.

17. The tool driver of claim 16, the torque sensing assembly including a magnetic torque sensing assembly.

18. The tool driver of claim 17, the magnetic torque sensing assembly including a tunneling magnetoresistance torque sensing assembly.

19. A robotic surgical system comprising:
the tool driver of claim 16; and
a surgical instrument including:
an instrument base,
an elongate shaft extending distally from the instrument base, and
a drive input movably mounted to the instrument base,
the drive output of the tool driver being configured to operatively engage the drive input of the surgical instrument.

20. A tool driver for a robotic surgical system, the tool driver comprising:
a housing;
a drive output movably mounted to the housing and configured to operatively engage a drive input of a surgical instrument;
an actuator including:
a housing assembly including:
an internal ring gear centered relative to a central axis, and
a top cover including:
a first rigid portion fixedly secured against movement relative to the internal ring gear,
a second rigid portion fixedly secured against movement relative to the housing of the tool driver, and
at least one flexible bridge extending between the first rigid portion and the second rigid portion,
a cycloid disc positioned eccentrically relative to the central axis, the cycloid disc including an external ring gear configured to selectively engage the internal ring gear,
a rotor configured to drive eccentric movement of the cycloid disc relative to the central axis,
an eccentric coupler configured to convert eccentric movement of the cycloid disc relative to the central axis into rotation of the drive output about the central axis; and
a torque sensing assembly configured to detect relative rotational movement between the first rigid portion and the second rigid portion about the central axis.

21-40. (canceled)
